(19) United States
(12) Patent Application Publication
(10) Pub. No.: US 2025/0261639 A1
(43) Pub. Date: Aug. 21, 2025

(54) ZEALACTONE DERIVATIVES AS PLANT GROWTH REGULATORS

(71) Applicant: SYNGENTA CROP PROTECTION AG, Basel (CH)

(72) Inventors: Alain DE MESMAEKER, Stein (CH); Michael Christian DIECKMANN, Münchwilen (CH); Pierre QUINODOZ, Stein (CH); Masahiko YOSHIMURA, Kyoto (JP)

(73) Assignee: SYNGENTA CROP PROTECTION AG, Basel (CH)

(21) Appl. No.: 18/249,834

(22) PCT Filed: Oct. 19, 2021
(86) PCT No.: PCT/EP2021/078997

§ 371 Date: Apr. 20, 2023

(30) Foreign Application Priority Data
Oct. 22, 2020 (EP) 20203366.8

(51) Int. Cl. A01N 43/08; A01C 1/06; A01G 7/06; A01P 21/00; C07D 307/60

(52) U.S. Cl. CPC A01N 43/08; A01C 1/06; A01G 7/06; A01P 21/00; C07D 307/60

(57) ABSTRACT

Compounds of the formula (I) wherein the substituents are as defined in claim 1, useful as plant growth regulators and/or seed germination promoters.

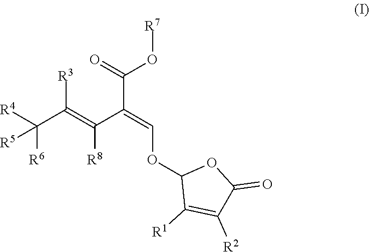

### CROSS-REFERENCE TO RELATED APPLICATIONS

This application is a U.S. National Stage Application, filed under 35 U.S.C. § 371, of International Application No. PCT/EP2021/078997, filed Oct. 19, 2021, which claims priority to EP 20203366.8, filed Oct. 22, 2020, the entire contents of which are incorporated by reference herein.

The present invention relates to novel zealactone derivatives, to processes for preparing these derivatives including intermediate compounds, to seeds comprising these derivatives, to plant growth regulator or seed germination promoting compositions comprising these derivatives and to methods of using these derivatives in controlling the growth of plants, improving the nutrient use efficiency of plants, and/or promoting the germination of seeds.

Zealactone per se is known from Xiaonan Xie, Takaya Kisugi, Kaori Yoneyama et al, ‘Methyl zealactonoate, a novel germination stimulant for root parasitic weeds produced by maize’, Journal of Pesticile Science 2017, 42, 58-61, and T. V. Charnikhova, K. Gaus, A. Lumbroso et al, ‘Zealactones. Novel natural strigolactones from maize’, Phytochemistry 2017, 137, 123-131, and a synthetic approach to zealactone is known from M. Yoshimura, M. Dieckmann, P-Y. Dakas et al. ‘Total Synthesis and Biological Evaluation of Zealactone 1a/b’, Helv. Chim. Acta 2020, 103, e2000017.

According to the present invention, there is provided a compound of formula (I):

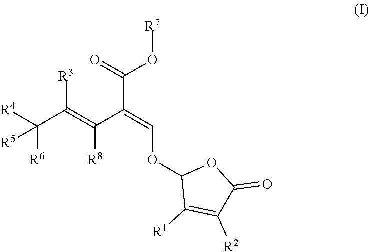

wherein
R¹ is hydrogen, halogen, C₁-C₃alkyl, C₁-C₃alkoxy, or C₃-C₆cycloalkyl;
R² is hydrogen, halogen, C₁-C₃alkyl, C₁-C₃alkoxy, or C₃-C₆cycloalkyl;
R³ is hydrogen or C₁-C₃alkyl;
R⁴ is hydrogen, cyano, C₁-C₆alkyl, C₁-C₆alkoxy, C₁-C₆haloalkyl, C₂-C₆alkenyl, C₂-C₆alkynyl, C₁-C₃alkoxycarbonyl, N,N-di(C₁-C₃alkyl)aminocarbonyl, N,N-di(C₁-C₃alkyl)aminooxycarbonyl, C₁-C₆alkylcarbonyl-N(C₁-C₃alkyl)amino, C₁-C₆alkylsulfinyl, C₁-C₆alkylsulfonyl, or C₁-C₆alkylsulfanyl;
R⁵ is hydrogen, C₁-C₆alkyl, C₁-C₆alkoxy, C₁-C₆haloalkyl, C₂-C₆alkenyl, or C₂-C₆alkynyl;
R⁶ is hydrogen, cyano, C₁-C₆alkyl, C₁-C₆alkoxy, C₁-C₆haloalkyl, C₂-C₆alkenyl, C₂-C₆alkynyl, C₁-C₃alkoxycarbonyl, N,N-di(C₁-C₃alkyl)aminocarbonyl, N,N-di(C₁-C₃alkyl)aminooxycarbonyl, C₁-C₆alkylcarbonyl-N(C₁-C₃alkyl)amino, C₁-C₆alkylsulfinyl, C₁-C₆alkylsulfonyl, or C₁-C₆alkylsulfanyl; or
R⁴ and R⁶ together with the carbon atom to which they are attached form an oxo group or a 3- to 6-membered cycloalkyl ring, optionally substituted with 1, 2, 3, or 4 groups selected from R⁹; or
R⁵ and R⁶ together with the carbon atom to which they are attached form an oxo group or a 3- to 6-membered cycloalkyl ring, optionally substituted with 1, 2, 3, or 4 groups selected from R⁹; or
R⁴, R⁵ and R⁶ together with the carbon atom to which they are attached form an 8- to 12-membered carbotricyclic ring system optionally substituted with 1, 2, 3, or 4 groups selected from R⁹;
R⁷ is hydrogen, C₁-C₆alkyl, C₁-C₆haloalkyl, or C₃-C₆cycloalkyl;
R⁸ is hydrogen or C₁-C₃alkyl;
R⁹ is halogen, C₁-C₃alkyl, C₁-C₃alkoxy, cyano, or oxo (═O);
or a salt thereof.

In a second aspect of the invention, there is provided a plant growth regulating or seed germination promoting composition, comprising the compound according to the present invention, and optionally, an agriculturally acceptable formulation adjuvant.

In a third aspect of the invention, there is provided a method for regulating the growth of plants at a locus, wherein the method comprises applying to the locus a plant growth regulating amount of the composition according to the second aspect of the invention.

In a fourth aspect of the invention, there is provided a method for promoting the germination of seeds comprising applying to the seeds, or a locus containing seeds, a seed germination promoting amount of a composition according to the second aspect of the invention.

In a fifth aspect of the invention, there is provided a method for controlling weeds, comprising applying to a locus containing weed seeds, a seed germination promoting amount of a composition according to the second aspect of the invention, allowing the seeds to germinate, and then applying to the locus a post-emergence herbicide.

In a sixth aspect of the invention, there is provided the use of a compound of formula (I) according to the invention as a plant growth regulator or a seed germination promoter.

In a seventh aspect of the invention, there is provided a method of treating a plant propagation material comprising applying to the plant propagation material a compound or composition according to the invention in an amount effective to promote germination and/or regulate plant growth.

In an eighth aspect of the invention, there is provided a method for improving the nutrient uptake of a crop, comprising applying to the plant or locus thereof, a compound of formula (I) or composition according to the invention in an amount effective to promote nutrient uptake.

In a ninth aspect of the invention, there is provided a plant propagation material treated with a compound of formula (I) according to the invention, or a composition according to the invention.

In a tenth aspect of the invention, there is provided a seed comprising a compound of formula (I) according to the invention.

Where substituents are indicated as being “optionally substituted”, this means that they may or may not carry one or more identical or different substituents, e.g., one, two or three R⁹ substituents. For example, C₁-C₆alkyl substituted by 1, 2 or 3 halogens, may include, but not be limited to, —CH₂Cl, —CHCl₂, —CCl₃, —CH₂F, —CHF₂, —CF₃, —CH₂CF₃ or —CF₂CH₃ groups. As another example, C₁-C₆alkoxy substituted by 1, 2 or 3 halogens, may include, but not limited to, CH₂ClO—, CHCl₂O—, CCl₃O—, CH₂FO—, CHF₂O—, CF₃O—, CF₃CH₂O— or CH₃CF₂O— groups.

As used herein, the term “cyano” means a —CN group.

As used herein, the term “halogen” refers to fluorine (fluoro), chlorine (chloro), bromine (bromo) or iodine (iodo).

As used herein, ═O means an oxo group, e.g., as found in a carbonyl (—C(═O)—) group.

As used herein, the term “C₁-C₆alkyl” refers to a straight or branched hydrocarbon chain radical consisting solely of carbon and hydrogen atoms, containing no unsaturation, having from one to six carbon atoms, and which is attached to the rest of the molecule by a single bond. “C₁-C₄alkyl” and “C₁-C₃alkyl” are to be construed accordingly. Examples of C₁-C₆alkyl include, but are not limited to, methyl, ethyl, n-propyl, and the isomers thereof, for example, iso-propyl. A “C₁-C₆alkylene” group refers to the corresponding definition of C₁-C₆alkyl, except that such radical is attached to the rest of the molecule by two single bonds. The term “C₁-C₂alkylene” is to be construed accordingly. Examples of C₁-C₆alkylene, include, but are not limited to, —CH₂—, —CH₂CH₂— and —(CH₂)₃—.

As used herein, the term “C₁-C₆haloalkyl” refers to a C₁-C₆alkyl radical as generally defined above substituted by one or more of the same or different halogen atoms. The terms “C₁-C₄haloalkyl” and “C₁-C₃haloalkyl”, are to be construed accordingly. Examples of C₁-C₆haloalkyl include, but are not limited to trifluoromethyl.

As used herein, the term “cyanoC₁-C₆alkyl” refers to a C₁-C₆alkyl radical as generally defined above substituted by one or more cyano groups as defined above.

As used herein, the term “C₁-C₆alkoxy” refers to a radical of the formula —ORₐ where Rₐ is a C₁-C₆alkyl radical as generally defined above. The terms “C₁-C₄alkoxy” and “C₁-C₃alkoxy” are to be construed accordingly. Examples of C₁-C₆alkoxy include, but are not limited to, methoxy, ethoxy, 1-methylethoxy (iso-propoxy), and propoxy.

As used herein, the term “C₁-C₄haloalkoxy” refers to a radical of the formula —ORₐ where Rₐ is a C₁-C₄alkyl radical as generally defined above substituted by one or more of the same or different halogen atoms.

As used herein, the term “C₂-C₆alkenyl” refers to a straight or branched hydrocarbon chain radical group consisting solely of carbon and hydrogen atoms, containing at least one double bond that can be of either the (E)- or (Z)-configuration, having from two to six carbon atoms, which is attached to the rest of the molecule by a single bond. “C₂-C₄alkenyl” and “C₂-C₃alkenyl” are to be construed accordingly. Examples of C₂-C₆alkenyl include, but are not limited to, ethenyl (vinyl), prop-1-enyl, prop-2-enyl (allyl), but-1-enyl.

As used herein, the term “C₂-C₆alkynyl” refers to a straight or branched hydrocarbon chain radical group consisting solely of carbon and hydrogen atoms, containing at least one triple bond, having from two to six carbon atoms, and which is attached to the rest of the molecule by a single bond. “C₂-C₄alkynyl” and “C₂-C₃alkynyl” are to be construed accordingly. Examples of C₂-C₆alkynyl include, but are not limited to, ethynyl, prop-1-ynyl, but-1-ynyl.

As used herein, the term “N,N-di(C₁-C₃alkyl)aminooxycarbonyl” refers to a radical of the formula (Rₐ)(R_{b})NCO(O)—, wherein Rₐ and R_{b} are each independently C₁-C₃alkyl radicals as generally defined above.

As used herein, the term “N,N-di(C₁-C₃alkyl)aminocarbonyl” refers to a radical of the formula (Rₐ)(R_{b})NC(O)—, wherein Rₐ and R_{b} are each independently C₁-C₃alkyl radicals as generally defined above. Examples of “N,N-di(C₁-C₃alkyl)aminocarbonyl” include, but are not limited to, N,N-di(methyl)aminocarbonyl.

As used herein, the term “C₁-C₆alkylcarbonyl-N(C₁-C₃alkyl)amino” refers to a radical of the formula RₐC(O)NH(R_{b})—, wherein Rₐ is a C₁-C₆alkyl radical as generally defined above, and R_{b} is a C₁-C₃alkyl radical as generally defined above. Examples of “C₁-C₆alkylcarbonyl-N(C₁-C₃alkyl)amino” include, but are not limited to, methylcarbonyl-N(methyl)amino.

As used herein, the term “C₃-C₆cycloalkyl” refers to a radical which is a monocyclic saturated ring system and which contains 3 to 6 carbon atoms. “C₃-C₅cycloalkyl” and “C₃-C₄cycloalkyl” are to be construed accordingly. Examples of C₃-C₆cycloalkyl include, but are not limited to, cyclopropyl, 1-methylcyclopropyl, 2-methylcyclopropyl, cyclobutyl, 1-methylcyclobutyl, 1,1-dimethylcyclobutyl, 2-methylcyclobutyl, and 2,2-dimethylcyclobutyl.

As used herein, the term “C₁-C₃alkoxycarbonyl” refers to a radical of the formula —C(O)ORₐ, where Rₐ is a C₁-C₃alkyl radical as generally defined above.

As used herein, the term “C₁-C₆alkylsulfanyl” refers to a radical of the formula —SRₐ, where Rₐ is a C₁-C₆alkyl radical as generally defined above. The terms “C₁-C₄alkylsulfanyl” and “C₁-C₃alkylsulfanyl”, are to be construed accordingly. Examples of C₁-C₆alkylsulfanyl include, but are not limited to methylsulfanyl.

As used herein, the term “C₁-C₆alkylsulfinyl” refers to a radical of the formula —S(O)Rₐ, where Rₐ is a C₁-C₆alkyl radical as generally defined above. The terms “C₁-C₄alkylsulfinyl” and “C₁-C₃alkylsulfinyl”, are to be construed accordingly. Examples of C₁-C₆alkylsulfinyl include, but are not limited to methylsulfinyl.

As used herein, the term “C₁-C₆alkylsulfonyl” refers to a radical of the formula —S(O)₂Rₐ, where Rₐ is a C₁-C₆alkyl radical as generally defined above. The terms “C₁-C₄alkylsulfonyl” and “C₁-C₃alkylsulfonyl”, are to be construed accordingly. Examples of C₁-C₆alkylsulfanyl include, but are not limited to methylsulfonyl.

As used herein, a “carbotricyclic ring” refers to a non-aromatic tricyclic ring system comprising three rings joined together at one carbon atom, i.e., sharing one carbon atom. Examples of a carbotricyclic ring system include, but are not limited to, adamantyl.

The presence of one or more possible stereogenic elements in a compound of formula (I) means that the compounds may occur in optically isomeric forms, i.e., enantiomeric or diastereomeric forms. Also, atropisomers may occur as a result of restricted rotation about a single bond. Formula (I) is intended to include all those possible isomeric forms and mixtures thereof. The present invention includes all those possible isomeric forms and mixtures thereof for a compound of formula (I). Likewise, formula (I) is intended to include all possible tautomers. The present invention includes all possible tautomeric forms for a compound of formula (I).

In each case, the compounds of formula (I) according to the invention are in free form, or in salt form, e.g., an agronomically usable salt form.

The following list provides definitions, including preferred definitions, for substituents R¹, R², R³, R⁴, R⁵, R⁶, R⁷, R⁸, and R⁹ with reference to compounds of formula (I). For any one of these substituents, any of the definitions given below may be combined with any definition of any other substituent given below or elsewhere in this document.

R¹ is hydrogen, halogen, C₁-C₃alkyl, C₁-C₃alkoxy, or C₃-C₆cycloalkyl. Preferably, R¹ is hydrogen, halogen, C₁-C₃alkyl, C₁-C₃alkoxy, or C₃-C₄cycloalkyl. More preferably, R¹ is hydrogen, halogen, C₁-C₃alkyl, or C₁-C₃alkoxy. Even more preferably, R¹ is hydrogen, C₁-C₃alkyl, or C₁-C₃alkoxy. More preferably still, R¹ is hydrogen, methyl, or methoxy. Even more preferably still, R¹ is hydrogen or methyl. In one embodiment, R¹ is hydrogen.

R² is hydrogen, halogen, C₁-C₃alkyl, C₁-C₃alkoxy, or C₃-C₆cycloalkyl. Preferably, R² is hydrogen, halogen, C₁-C₃alkyl, C₁-C₃alkoxy, or C₃-C₄cycloalkyl. More preferably, R² is hydrogen, halogen, C₁-C₃alkyl, or C₁-C₃alkoxy. Even more preferably, R² is hydrogen, C₁-C₃alkyl, or C₁-C₃alkoxy. More preferably still, R² is hydrogen or C₁-C₃alkyl. Even more preferably still, R² is hydrogen or methyl. In one embodiment, R² is C₁-C₃alkyl, preferably methyl. In another embodiment, R² is C₁-C₃alkyl or C₁-C₃alkoxy, preferably, methyl or methoxy.

In one set of embodiments, when R¹ is hydrogen, R² is methyl. In another set of embodiments, R¹ and R² are both methyl.

R³ is hydrogen or C₁-C₃alkyl. Preferably, R³ is hydrogen, methyl, ethyl, or isopropyl. More preferably still, R³ is hydrogen or methyl. In one embodiment, R³ is hydrogen.

R⁴ is hydrogen, cyano, C₁-C₆alkyl, C₁-C₆alkoxy, C₁-C₆haloalkyl, C₂-C₆alkenyl, C₂-C₆alkynyl, C₁-C₃alkoxycarbonyl, N,N-di(C₁-C₃alkyl)aminocarbonyl, N,N-di(C₁-C₃alkyl)aminooxycarbonyl, C₁-C₆alkylcarbonyl-N(C₁-C₃alkyl)amino, C₁-C₆alkylsulfinyl, C₁-C₆alkylsulfonyl, or C₁-C₆alkylsulfanyl.

Preferably, R⁴ is hydrogen, cyano, C₁-C₆alkyl, C₁-C₆alkoxy, C₁-C₆haloalkyl, C₂-C₆alkenyl, C₂-C₆alkynyl, C₁-C₃alkoxycarbonyl, N,N-di(C₁-C₃alkyl)aminocarbonyl, N,N-di(C₁-C₆alkyl)aminooxycarbonyl, C₁-C₃alkylcarbonyl-N(C₁-C₃alkyl)amino, C₁-C₃alkylsulfinyl, C₁-C₃alkylsulfonyl, or C₁-C₃alkylsulfanyl.

More preferably, R⁴ is hydrogen, cyano, C₁-C₃alkyl, C₁-C₃alkoxy, C₁-C₃haloalkyl, C₂-C₄alkenyl, C₁-C₃alkoxycarbonyl, N,N-di(C₁-C₃alkyl)aminocarbonyl, C₁-C₃alkylcarbonyl-N(C₁-C₃alkyl)amino, or C₁-C₃alkylsulfonyl.

More preferably still, R⁴ is hydrogen, cyano, methyl, ethyl, n-propyl, isopropyl, methoxy, ethoxy, n-propoxy, isopropoxy, trifluoromethyl, difluoromethyl, 2,2,2-trifluoroethyl, ethenyl, propenyl, 1-isopropenyl, methoxycarbonyl, ethoxycarbonyl, isopropoxycarbonyl, N,N-di(methyl)aminocarbonyl, methylcarbonyl-N(methyl)amino, or methylsulfonyl, ethylsulfonyl, or isopropylsulfonyl.

In one set of embodiments, R⁴ is hydrogen, cyano, C₁-C₃alkyl, C₁-C₃alkoxy, C₁-C₃alkoxycarbonyl, N,N-di(C₁-C₂alkyl)aminocarbonyl, C₁-C₂alkylcarbonyl-N(C₁-C₂alkyl)amino, or C₁-C₃alkylsulfonyl More preferably, R⁴ is hydrogen, cyano, methyl, methoxy, methoxycarbonyl, N,N-di(methyl)aminocarbonyl, methylcarbonyl-N(methyl)amino, or methylsulfonyl.

In one set of embodiments, R⁴ is hydrogen, cyano, C₁-C₃alkyl, C₁-C₃alkoxy, C₁-C₃haloalkyl, C₂-C₄alkenyl, C₁-C₃alkoxycarbonyl, N,N-di(methyl)aminocarbonyl, N,N-di(methyl)aminooxycarbonyl, C₁-C₃alkylcarbonyl-N(methyl)amino, or C₁-C₃alkylsulfonyl.

In another set of embodiments, R⁴ is hydrogen, cyano, C₁-C₆alkyl, C₁-C₆alkoxy, C₁-C₆haloalkyl, C₂-C₆alkenyl, C₂-C₆alkynyl, C₁-C₃alkoxycarbonyl, N,N-di(C₁-C₃alkyl)aminooxycarbonyl, C₁-C₆alkylsulfinyl, C₁-C₆alkylsulfonyl, or C₁-C₆alkylsulfanyl. Preferably, R⁴ is hydrogen, cyano, C₁-C₄alkyl, C₁-C₄alkoxy, C₁-C₄haloalkyl, C₂-C₄alkenyl, C₂-C₄alkynyl, C₁-C₃alkoxycarbonyl, N,N-di(C₁-C₃alkyl)aminooxycarbonyl, C₁-C₄alkylsulfinyl, C₁-C₄alkylsulfonyl, or C₁-C₄alkylsulfanyl. More preferably, R⁴ is hydrogen, cyano, C₁-C₄alkyl, C₁-C₄alkoxy, C₁-C₄haloalkyl, C₂-C₄alkenyl, C₂-C₄alkynyl, C₁-C₄alkylsulfonyl, or C₁-C₄alkylsulfanyl. More preferably, R⁴ is hydrogen, cyano, C₁-C₃alkyl, C₁-C₃alkoxy, C₁-C₃haloalkyl, C₁-C₃alkylsulfonyl, or C₁-C₃alkylsulfanyl. More preferably still, R⁴ is hydrogen, cyano, methyl, ethyl, methoxy, methylsulfanyl, or methylsulfonyl. Even more preferably still, R⁴ is hydrogen or methyl.

R⁵ is hydrogen, C₁-C₆alkyl, C₁-C₆alkoxy, C₁-C₆haloalkyl, C₂-C₆alkenyl, or C₂-C₆alkynyl. Preferably, R⁵ is hydrogen, C₁-C₄alkyl, C₁-C₄alkoxy, C₁-C₄haloalkyl, C₂-C₅alkenyl, or C₂-C₅alkynyl. More preferably, R⁵ is hydrogen, C₁-C₃alkyl, or C₁-C₃alkoxy. More preferably still, R⁵ is hydrogen, methyl, or methoxy. Even more preferably still, R⁵ is hydrogen or methyl.

In one set of embodiments, R⁵ is hydrogen, C₁-C₆alkyl, or C₂-C₆alkenyl, preferably, hydrogen, C₁-C₃alkyl, or C₂-C₄alkenyl, more preferably, hydrogen, C₁-C₃alkyl, or C₂-C₃alkenyl, and even more preferably, hydrogen, methyl, or isopropenyl.

R⁶ is hydrogen, cyano, C₁-C₆alkyl, C₁-C₆alkoxy, C₁-C₆haloalkyl, C₂-C₆alkenyl, C₂-C₆alkynyl, C₁-C₃alkoxycarbonyl, N,N-di(C₁-C₃alkyl)aminocarbonyl, N,N-di(C₁-C₃alkyl)aminooxycarbonyl, C₁-C₆alkylcarbonyl-N(C₁-C₃alkyl)amino, C₁-C₆alkylsulfinyl, C₁-C₆alkylsulfonyl, or C₁-C₆alkylsulfanyl.

Preferably, R⁶ is hydrogen, cyano, C₁-C₆alkyl, C₁-C₆alkoxy, C₁-C₆haloalkyl, C₂-C₆alkenyl, C₂-C₆alkynyl, C₁-C₃alkoxycarbonyl, N,N-di(C₁-C₃alkyl)aminocarbonyl, N,N-di(C₁-C₃alkyl)aminooxycarbonyl, C₁-C₃alkylcarbonyl-N(C₁-C₃alkyl)amino, C₁-C₃alkylsulfinyl, C₁-C₃alkylsulfonyl, or C₁-C₃alkylsulfanyl.

More preferably, R⁶ is hydrogen, cyano, C₁-C₃alkyl, C₁-C₃alkoxy, C₁-C₃haloalkyl, C₂-C₄alkenyl, C₁-C₃alkoxycarbonyl, N,N-di(C₁-C₃alkyl)aminocarbonyl, C₁-C₃alkylcarbonyl-N(C₁-C₃alkyl)amino, or C₁-C₃alkylsulfonyl.

More preferably still, R⁶ is hydrogen, cyano, methyl, ethyl, n-propyl, isopropyl, methoxy, ethoxy, n-propoxy, isopropoxy, trifluoromethyl, difluoromethyl, 2,2,2-trifluoroethyl, ethenyl, propenyl, 1-isopropenyl, methoxycarbonyl, ethoxycarbonyl, isopropoxycarbonyl, N,N-di(methyl)aminocarbonyl, methylcarbonyl-N(methyl)amino, or methylsulfonyl, ethylsulfonyl, or isopropylsulfonyl.

In one set of embodiments, R⁶ is hydrogen, cyano, C₁-C₃alkyl, C₁-C₃alkoxy, C₁-C₃alkoxycarbonyl, N,N-di(C₁-C₂alkyl)aminocarbonyl, C₁-C₂alkylcarbonyl-N(C₁-C₂alkyl)amino, or C₁-C₃alkylsulfonyl.

More preferably, R⁶ is hydrogen, cyano, methyl, methoxy, methoxycarbonyl, N,N-di(methyl)aminocarbonyl, methylcarbonyl-N(methyl)amino, or methylsulfonyl

In another set of embodiments, R⁶ is hydrogen or C₁-C₆alkyl, preferably, hydrogen or C₁-C₃alkyl, and more preferably, hydrogen or methyl.

In one set of embodiments, R⁶ is hydrogen, cyano, C₁-C₆alkyl, C₁-C₆alkoxy, C₁-C₆haloalkyl, C₂-C₆alkenyl, C₂-C₆alkynyl, C₁-C₃alkoxycarbonyl, N,N-di(C₁-C₃alkyl)aminooxycarbonyl, C₁-C₆alkylsulfinyl, C₁-C₆alkylsulfonyl, or C₁-C₆alkylsulfanyl; Preferably, R⁶ is hydrogen, cyano, C₁-C₄alkyl, C₁-C₄alkoxy, C₁-C₄haloalkyl, C₂-C₄alkenyl, C₂-C₄alkynyl, C₁-C₃alkoxycarbonyl, N,N-di(C₁-C₃alkyl)aminooxycarbonyl, C₁-C₄alkylsulfinyl, C₁-C₄alkylsulfonyl, or C₁-C₄alkylsulfanyl. More preferably, R⁶ is hydrogen, cyano, C₁-C₃alkyl, C₁-C₃alkoxy, C₁-C₃alkoxycarbonyl, N,N-di(C₁-C₃alkyl)aminooxycarbonyl, C₁-C₃alkylsulfonyl, or C₁-C₃alkylsulfanyl. Even more preferably, R⁶ is hydrogen, cyano, methyl, methoxy, methoxycarbonyl, N,N-di(methyl)aminooxycarbonyl, or methylsulfonyl.

Or R⁴ and R⁶ together with the carbon atom to which they are attached form an oxo (═O) group or a 3- to 6-membered cycloalkyl ring optionally substituted with 1, 2, 3, or 4 groups selected from R⁹. Preferably, R⁴ and R⁶ together with the carbon atom to which they are attached form an oxo group or a 3- to 6-membered cycloalkyl ring optionally substituted with 1, 2, or 3 groups selected from R⁹. More preferably, R⁴ and R⁶ together with the carbon atom to which they are attached form an oxo group or a 3- to 6-membered cycloalkyl ring optionally substituted with 1 or 2 groups selected from R⁹.

In one set of embodiments, R⁴ and R⁶ together with the carbon atom to which they are attached form a 3- to 6-membered cycloalkyl ring optionally substituted with 1, 2, 3, or 4 groups selected from R⁹-more preferably, R⁴ and R⁶ together with the carbon atom to which they are attached form a 4- to 6-membered cycloalkyl ring optionally substituted with 1, 2, 3, or 4 groups selected from R⁹; even more preferably, R⁴ and R⁶ together with the carbon atom to which they are attached form a 4- to 6-membered cycloalkyl ring optionally substituted with 1, 2, or 3 groups selected from R⁹; more preferably still, R⁴ and R⁶ together with the carbon atom to which they are attached form a 4- to 6-membered cycloalkyl ring, or a 4- or 6-membered cycloalkyl ring substituted with 2 or 3 groups selected from R⁹.

In a particularly preferred set of embodiments, R⁴ and R⁶ together with the carbon atom to which they are attached form a cyclobutyl, cyclopentyl, or cyclohexyl ring, wherein the cyclobutyl ring is optionally substituted with 3 groups selected from R⁹, and wherein the cyclohexyl ring is optionally substituted with 2 groups selected from R⁹.

Or R⁵ and R⁶ together with the carbon atom to which they are attached form an oxo group or a 3- to 6-membered cycloalkyl ring, optionally substituted with 1, 2, 3, or 4 groups selected from R⁹.

Or R⁴, R⁵ and R⁶ together with the carbon atom to which they are attached form an 8- to 12-membered carbotricyclic ring system optionally substituted with 1, 2, 3, or 4 groups selected from R⁹. Preferably, R⁴, R⁵ and R⁶ together with the carbon atom to which they are attached form an 8- to 10-membered carbotricyclic ring system optionally substituted with 1, 2, or 3 groups selected from R⁹. More preferably, R⁴, R⁵ and R⁶ together with the carbon atom to which they are attached form an 8- to 10-membered carbotricyclic ring system optionally substituted with 1 or 2 groups selected from R⁹. More preferably still, R⁴, R⁵ and R⁶ together with the carbon atom to which they are attached form an 8- to 10-membered carbotricyclic ring system. Even more preferably, R⁴, R⁵ and R⁶ together with the carbon atom to which they are attached form an adamantyl ring.

R⁷ is hydrogen, C₁-C₆alkyl, C₁-C₆haloalkyl, or C₃-C₆cycloalkyl. Preferably, R⁷ is hydrogen, C₁-C₆alkyl, C₁-C₃haloalkyl, or C₃-C₆cycloalkyl. More preferably, R⁷ is hydrogen or C₁-C₃alkyl, even more preferably, R⁷ is C₁-C₃alkyl, and most preferably, R⁷ is methyl.

R⁸ is hydrogen or C₁-C₃alkyl. Preferably, R⁸ is hydrogen, methyl, or ethyl. More preferably, R⁸ is hydrogen or methyl. More preferably still, R⁸ is hydrogen.

R⁹ is halogen, C₁-C₃alkyl, C₁-C₃alkoxy, cyano, or oxo (═O). Preferably, R⁹ is chloro, fluoro, methyl, methoxy, cyano, or oxo (═O). In one set of embodiments, R⁹ is halogen, C₁-C₃alkyl, or oxo (═O), preferably, halogen, methyl, or oxo (═O), more preferably, fluoro, methyl, or oxo (═O). In another set of embodiments, R⁹ is halogen, preferably fluoro.

In a compound of formula (I) according to the present invention, preferably:
R¹ is hydrogen, C₁-C₃alkyl, or C₁-C₃alkoxy;
R² is hydrogen, C₁-C₃alkyl, or C₁-C₃alkoxy;
R³ is hydrogen or C₁-C₃alkyl;
R⁴ is hydrogen, cyano, C₁-C₃alkyl, C₁-C₃alkoxy, C₁-C₃haloalkyl, C₁-C₃alkylsulfonyl, or C₁-C₃alkylsulfanyl;
R⁵ is hydrogen, C₁-C₃alkyl, C₁-C₃alkoxy, or C₂-C₄alkenyl;
R⁶ is hydrogen, cyano, C₁-C₃alkyl, C₁-C₃alkoxy, C₁-C₃alkoxycarbonyl, N,N-di(C₁-C₃alkyl)aminooxycarbonyl, C₁-C₃alkylsulfonyl, or C₁-C₃alkylsulfanyl; or
R⁴ and R⁶ together with the carbon atom to which they are attached form an oxo group or a 3- to 6-membered cycloalkyl ring optionally substituted with 1, 2, or 3 groups selected from R⁹; or
R⁴, R⁵ and R⁶ together with the carbon atom to which they are attached form an 8- to 10-membered carbotricyclic ring system optionally substituted with 1 or 2 groups selected from R⁹;
R⁷ is C₁-C₃alkyl;
R⁸ is hydrogen or C₁-C₃alkyl; and
R⁹ is halogen.

In another set of embodiments, R¹ is hydrogen, methyl, or methoxy;
R² is hydrogen or methyl;
R³ is hydrogen or methyl;
R⁴ is hydrogen, cyano, methyl, ethyl, methoxy, methylsulfanyl, or methylsulfonyl;
R⁵ is hydrogen, methyl, or methoxy;
R⁶ is hydrogen, cyano, methyl, methoxy, methoxycarbonyl, N,N-di(methyl)aminooxycarbonyl, or methylsulfonyl; or
R⁴ and R⁶ together with the carbon atom to which they are attached form an oxo group or a 3- to 6-membered cycloalkyl ring optionally substituted with 1 or 2 groups selected from R⁹; or
R⁴, R⁵ and R⁶ together with the carbon atom to which they are attached form an 8- to 10-membered carbotricyclic ring system;
R⁷ is C₁-C₃alkyl;
R⁸ is hydrogen or C₁-C₃alkyl; and
R⁹ is halogen.

In another set of embodiments, R¹ is hydrogen or C₁-C₃alkyl;
R² is C₁-C₃alkyl;
R³ is hydrogen or C₁-C₃alkyl;
R⁴ is hydrogen, cyano, C₁-C₃alkyl, C₁-C₃alkoxy, C₁-C₃alkoxycarbonyl, N,N-di(C₁-C₃alkyl)aminocarbonyl, C₁-C₃alkylcarbonyl-N(C₁-C₃alkyl)amino, or C₁-C₃alkylsulfonyl;
R⁵ is hydrogen, C₁-C₃alkyl, or C₂-C₄alkenyl;
R⁶ is hydrogen or C₁-C₃alkyl; or
R⁴ and R⁶ together with the carbon atom to which they are attached form a 4- to 6-membered cycloalkyl ring, optionally substituted with 1, 2, or 3 groups selected from R⁹; or
R⁴, R⁵ and R⁶ together with the carbon atom to which they are attached form an adamantyl group;
R⁷ is methyl;
R⁸ is hydrogen; and
R⁹ is fluoro, methyl, or oxo (═O).

In a further set of embodiments, R¹ is hydrogen or C₁-C₃alkyl;
R² is C₁-C₃alkyl;
R³ is hydrogen or C₁-C₃alkyl;
R⁴ is hydrogen, cyano, methyl, methoxy, methoxycarbonyl, methylcarbonyl-N-(methyl)amino, N,N-di(methyl)-aminocarbonyl, or methylsulfonyl;
R⁵ is hydrogen, methyl, or isopropenyl;
R⁶ is hydrogen or methyl; or
R⁴ and R⁶ together with the carbon atom to which they are attached form a cyclobutyl, cyclopentyl, or cyclohexyl ring, optionally substituted with 1, 2, or 3 groups selected from fluoro, methyl, and oxo (═O); or
R⁴, R⁵ and R⁶ together with the carbon atom to which they are attached form an adamantyl group;
R⁷ is C₁-C₃alkyl; and
R⁸ is hydrogen.

In a still further set of embodiments, R¹ is hydrogen or methyl;
R² is methyl;
R³ is hydrogen or methyl;
R⁴ is hydrogen, cyano, methyl, methoxy, methoxycarbonyl, methylcarbonyl-N-(methyl)amino, N,N-di(methyl)-aminocarbonyl, or methylsulfonyl;
R⁵ is hydrogen, methyl, or isopropenyl;
R⁶ is hydrogen or methyl; or
R⁴ and R⁶ together with the carbon atom to which they are attached form a cyclobutyl, cyclopentyl, or cyclohexyl ring, optionally substituted with 1, 2, or 3 groups selected from fluoro, methyl, and oxo (═O); or
R⁴, R⁵ and R⁶ together with the carbon atom to which they are attached form an adamantyl group;
R⁷ is methyl; and
R⁸ is hydrogen.

In a particularly preferred set of embodiments, the compound of formula (I) is selected from:
methyl (E,2E)-4-(1-methylcyclohexyl)-2-[(4-methyl-5-oxo-2H-furan-2-yl)oxymethylene]but-3-enoate (P-1);
methyl (E,2E)-4-(4,4-difluorocyclohexyl)-2-[(4-methyl-5-oxo-2H-furan-2-yl)oxymethylene]but-3-enoate (P-2);
methyl (E,2E)-4-cyclohexyl-2-[(3,4-dimethyl-5-oxo-2H-furan-2-yl)oxymethylene]but-3-enoate (P-3);
methyl (E,2E)-4-(1-adamantyl)-2-[(4-methyl-5-oxo-2H-furan-2-yl)oxymethylene]but-3-enoate (P-4);
methyl (E,2E)-4-cyclobutyl-2-[(4-methyl-5-oxo-2H-furan-2-yl)oxymethylene]but-3-enoate (P-5);
methyl (E,2E)-4-(1-methylcyclopentyl)-2-[(4-methyl-5-oxo-2H-furan-2-yl)oxymethylene]but-3-enoate (P-6);
methyl (E,2E)-4-(1-methylcyclopropyl)-2-[(4-methyl-5-oxo-2H-furan-2-yl)oxymethylene]but-3-enoate (P-7);
dimethyl (E,4E)-4-[(4-methyl-5-oxo-2H-furan-2-yl)oxymethylene]pent-2-enedioate (P-8);
methyl (2E)-4-methyl-2-[(4-methyl-5-oxo-2H-furan-2-yl)oxymethylene]pent-3-enoate (P-9);
methyl (E,2E)-2-[(3,4-dimethyl-5-oxo-2H-furan-2-yl)oxymethylene]-5,5-dimethyl-hex-3-enoate (P-10);
methyl (2E,3E)-4-cyclopentyl-2-(((4-methyl-5-oxo-2,5-dihydrofuran-2-yl)oxy)methylene) but-3-enoate (P-11);
methyl (E,2E)-5-methoxy-5-methyl-2-[(4-methyl-5-oxo-2H-furan-2-yl)oxymethylene]hex-3-enoate (P-12);
methyl (E,2E)-4-cyclohexyl-2-[(4-methyl-5-oxo-2H-furan-2-yl)oxymethylene]but-3-enoate (P-13);
methyl (E,2E)-5,5-dimethyl-2-[(4-methyl-5-oxo-2H-furan-2-yl)oxymethylene]hex-3-enoate (P-14);
methyl (E,2E)-4-(1-isopropenyl-2,2-dimethyl-4-oxo-cyclobutyl)-2-[(4-methyl-5-oxo-2H-furan-2-yl)oxymethylene]but-3-enoate (P-15);
methyl (E,2E)-5-cyano-5-methyl-2-[(4-methyl-5-oxo-2H-furan-2-yl)oxymethylene]hex-3-enoate (P-16);
methyl (E,2E)-5-[acetyl(methyl)amino]-5-methyl-2-[(4-methyl-5-oxo-2H-furan-2-yl)oxymethylene]hex-3-enoate (P-17);
methyl (E,2E)-5-methyl-2-[(4-methyl-5-oxo-2H-furan-2-yl)oxymethylene]-5-methylsulfonyl-hex-3-enoate (P-18);
dimethyl (E,5E)-2,2-dimethyl-5-[(4-methyl-5-oxo-2H-furan-2-yl)oxymethylene]hex-3-enedioate (P-19); and
methyl (E,2E)-6-(dimethylamino)-5,5-dimethyl-2-[(4-methyl-5-oxo-2H-furan-2-yl)oxymethylene]-6-oxo-hex-3-enoate (P-20).

In another particularly preferred set of embodiments, the compound of formula (I) is selected from:
methyl (E,2E)-4-(4,4-difluorocyclohexyl)-2-[(4-methyl-5-oxo-2H-furan-2-yl)oxymethylene]but-3-enoate (P-2);
methyl (E,2E)-4-cyclohexyl-2-[(3,4-dimethyl-5-oxo-2H-furan-2-yl)oxymethylene]but-3-enoate (P-3);
methyl (E,2E)-4-(1-adamantyl)-2-[(4-methyl-5-oxo-2H-furan-2-yl)oxymethylene]but-3-enoate (P-4);
methyl (E,2E)-4-(1-methylcyclopentyl)-2-[(4-methyl-5-oxo-2H-furan-2-yl)oxymethylene]but-3-enoate (P-6);
dimethyl (E,4E)-4-[(4-methyl-5-oxo-2H-furan-2-yl)oxymethylene]pent-2-enedioate (P-8);
methyl (2E)-4-methyl-2-[(4-methyl-5-oxo-2H-furan-2-yl)oxymethylene]pent-3-enoate (P-9);
methyl (E,2E)-2-[(3,4-dimethyl-5-oxo-2H-furan-2-yl)oxymethylene]-5,5-dimethyl-hex-3-enoate (P-10);
methyl (E,2E)-4-(1-isopropenyl-2,2-dimethyl-4-oxo-cyclobutyl)-2-[(4-methyl-5-oxo-2H-furan-2-yl)oxymethylene]but-3-enoate (P-15); and
dimethyl (E,5E)-2,2-dimethyl-5-[(4-methyl-5-oxo-2H-furan-2-yl)oxymethylene]hex-3-enedioate (P-19).

In another particularly preferred set of embodiments, the compound of formula (I) is selected from:
methyl (E,2E)-4-(4,4-difluorocyclohexyl)-2-[(4-methyl-5-oxo-2H-furan-2-yl)oxymethylene]but-3-enoate (P-2);
methyl (E,2E)-4-(1-adamantyl)-2-[(4-methyl-5-oxo-2H-furan-2-yl)oxymethylene]but-3-enoate (P-4);
methyl (E,2E)-4-(1-methylcyclopentyl)-2-[(4-methyl-5-oxo-2H-furan-2-yl)oxymethylene]but-3-enoate (P-6);
methyl (E,2E)-4-(1-methylcyclopropyl)-2-[(4-methyl-5-oxo-2H-furan-2-yl)oxymethylene]but-3-enoate (P-7);
dimethyl (E,4E)-4-[(4-methyl-5-oxo-2H-furan-2-yl)oxymethylene]pent-2-enedioate (P-8);
methyl (E,2E)-5-methoxy-5-methyl-2-[(4-methyl-5-oxo-2H-furan-2-yl)oxymethylene]hex-3-enoate (P-12);
methyl (E,2E)-4-(1-isopropenyl-2,2-dimethyl-4-oxo-cyclobutyl)-2-[(4-methyl-5-oxo-2H-furan-2-yl)oxymethylene]but-3-enoate (P-15);
methyl (E,2E)-5-cyano-5-methyl-2-[(4-methyl-5-oxo-2H-furan-2-yl)oxymethylene]hex-3-enoate (P-16);
methyl (E,2E)-5-[acetyl(methyl)amino]-5-methyl-2-[(4-methyl-5-oxo-2H-furan-2-yl)oxymethylene]hex-3-enoate (P-17);
dimethyl (E,5E)-2,2-dimethyl-5-[(4-methyl-5-oxo-2H-furan-2-yl)oxymethylene]hex-3-enedioate (P-19); and
methyl (E,2E)-6-(dimethylamino)-5,5-dimethyl-2-[(4-methyl-5-oxo-2H-furan-2-yl)oxymethylene]-6-oxo-hex-3-enoate (P-20).

Compounds of the present invention can be made as shown in the following schemes, in which, unless otherwise stated, the definition of each variable is as defined above for a compound of formula (I).

Compounds of formula (I) may be prepared by coupling a compound of formula (II) wherein X is halogen with a compound of formula (III) wherein R is C₃-C₆alkyl and R³ and R⁸ are hydrogen, in the presence of a catalyst such as palladium (0). The reaction might be known by a person skilled in the art as a Stille coupling. This is shown in Scheme 1 below.

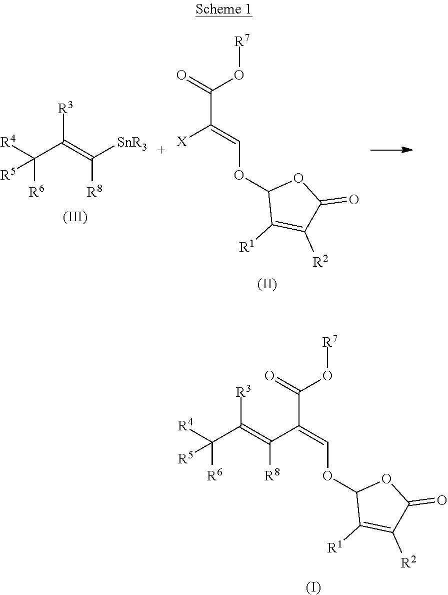

Alternatively, compounds of formula (I) may be prepared by coupling a compound of formula (IV) wherein R is C₃-C₆alkyl and R³ and R⁸ are hydrogen, with a compound of formula (V) wherein X is halogen in the presence of a catalyst such as palladium (0). The reaction might be known by a person skilled in the art as a Stille coupling. This is shown in Scheme 2 below.

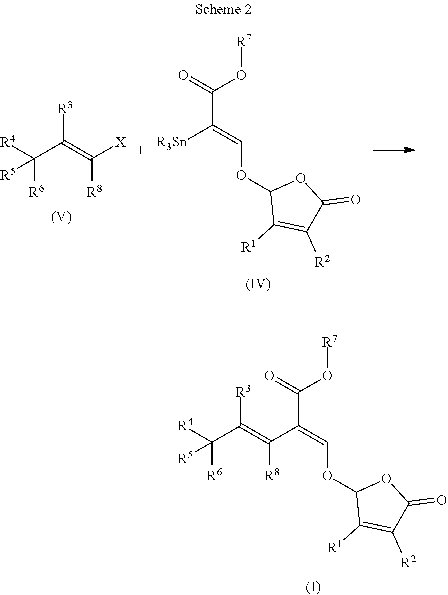

Compounds of formula (III) wherein R is C₃-C₆alkyl and R³ and R⁸ are hydrogen may be commercially available or may be prepared from compound of formula (VI) by reaction with R₃SnH in the presence of a catalyst such as Pd(0). Compounds of formula (VI) can be purchased from chemical suppliers. This is shown in Scheme 3 below.

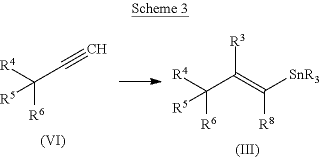

Compounds of formula (V) wherein X is halogen may be prepared by reaction of a compound of formula (III) with X₂ or any other electrophilic halogenating reagent. This is shown in Scheme 4 below.

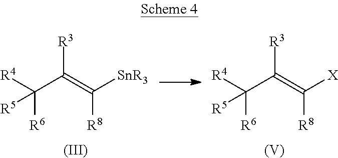

Compounds of formula (IV) may be prepared by coupling a compound of formula (II) wherein X is halogen with a compound of formula R₃Sn—SnR₃ in the presence of a catalyst such as Pd(0). This is shown in Scheme 5 below. Compounds of formula (II) may be prepared as described in M. Dieckmann et al., J. Org. Chem., 2018, 125-135.

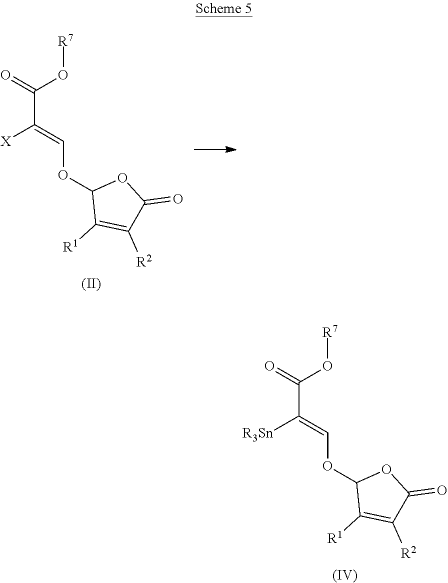

The tables below illustrate examples of individual compounds of formula (I) according to the invention.

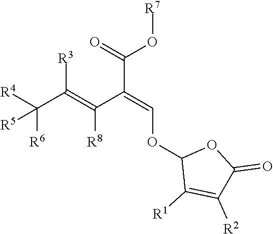

TABLE 1
Definitions for substituents R¹, R², R³, R⁵, R⁷, and R⁸ of
individual compounds of formula (I) according to the invention:

| Index | R¹ | R² | R³ | R⁵ | R⁷ | R⁸ |
|---|---|---|---|---|---|---|
| 001 | H | H | H | H | CH₃ | H |
| 002 | H | H | H | CH₃ | CH₃ | H |
| 003 | H | H | CH₃ | H | CH₃ | H |
| 004 | H | H | CH₃ | CH₃ | CH₃ | H |
| 005 | H | H | H | H | CH₃ | CH₃ |
| 006 | H | H | H | CH₃ | CH₃ | CH₃ |
| 007 | H | H | CH₃ | H | CH₃ | CH₃ |
| 008 | H | H | CH₃ | CH₃ | CH₃ | CH₃ |
| 009 | CH₃ | H | H | H | CH₃ | H |
| 010 | CH₃ | H | H | CH₃ | CH₃ | H |
| 011 | CH₃ | H | CH₃ | H | CH₃ | H |
| 012 | CH₃ | H | CH₃ | CH₃ | CH₃ | H |
| 013 | CH₃ | H | H | H | CH₃ | CH₃ |
| 014 | CH₃ | H | H | CH₃ | CH₃ | CH₃ |
| 015 | CH₃ | H | CH₃ | H | CH₃ | CH₃ |
| 016 | CH₃ | H | CH₃ | CH₃ | CH₃ | CH₃ |
| 017 | OCH₃ | H | H | H | CH₃ | H |
| 018 | OCH₃ | H | H | CH₃ | CH₃ | H |
| 019 | OCH₃ | H | CH₃ | H | CH₃ | H |
| 020 | OCH₃ | H | CH₃ | CH₃ | CH₃ | H |
| 021 | OCH₃ | H | H | H | CH₃ | CH₃ |
| 022 | OCH₃ | H | H | CH₃ | CH₃ | CH₃ |
| 023 | OCH₃ | H | CH₃ | H | CH₃ | CH₃ |
| 024 | OCH₃ | H | CH₃ | CH₃ | CH₃ | CH₃ |
| 025 | H | CH₃ | H | H | CH₃ | H |
| 026 | H | CH₃ | H | CH₃ | CH₃ | H |
| 027 | H | CH₃ | CH₃ | H | CH₃ | H |
| 028 | H | CH₃ | CH₃ | CH₃ | CH₃ | H |
| 029 | H | CH₃ | H | H | CH₃ | CH₃ |
| 030 | H | CH₃ | H | CH₃ | CH₃ | CH₃ |
| 031 | H | CH₃ | CH₃ | H | CH₃ | CH₃ |
| 032 | H | CH₃ | CH₃ | CH₃ | CH₃ | CH₃ |
| 033 | CH₃ | CH₃ | H | H | CH₃ | H |
| 034 | CH₃ | CH₃ | H | CH₃ | CH₃ | H |
| 035 | CH₃ | CH₃ | CH₃ | H | CH₃ | H |
| 036 | CH₃ | CH₃ | CH₃ | CH₃ | CH₃ | H |
| 037 | CH₃ | CH₃ | H | H | CH₃ | CH₃ |
| 038 | CH₃ | CH₃ | H | CH₃ | CH₃ | CH₃ |
| 039 | CH₃ | CH₃ | CH₃ | H | CH₃ | CH₃ |
| 040 | CH₃ | CH₃ | CH₃ | CH₃ | CH₃ | CH₃ |
| 041 | OCH₃ | CH₃ | H | H | CH₃ | H |
| 042 | OCH₃ | CH₃ | H | CH₃ | CH₃ | H |
| 043 | OCH₃ | CH₃ | CH₃ | H | CH₃ | H |
| 044 | OCH₃ | CH₃ | CH₃ | CH₃ | CH₃ | H |
| 045 | OCH₃ | CH₃ | H | H | CH₃ | CH₃ |
| 046 | OCH₃ | CH₃ | H | CH₃ | CH₃ | CH₃ |
| 047 | OCH₃ | CH₃ | CH₃ | H | CH₃ | CH₃ |
| 048 | OCH₃ | CH₃ | CH₃ | CH₃ | CH₃ | CH₃ |
| 049 | H | H | H | H | CH₂CH₃ | H |
| 050 | H | H | H | CH₃ | CH₂CH₃ | H |
| 051 | H | H | CH₃ | H | CH₂CH₃ | H |
| 052 | H | H | CH₃ | CH₃ | CH₂CH₃ | H |
| 053 | H | H | H | H | CH₂CH₃ | CH₃ |
| 054 | H | H | H | CH₃ | CH₂CH₃ | CH₃ |
| 055 | H | H | CH₃ | H | CH₂CH₃ | CH₃ |
| 056 | H | H | CH₃ | CH₃ | CH₂CH₃ | CH₃ |
| 057 | CH₃ | H | H | H | CH₂CH₃ | H |
| 058 | CH₃ | H | H | CH₃ | CH₂CH₃ | H |
| 059 | CH₃ | H | CH₃ | H | CH₂CH₃ | H |
| 060 | CH₃ | H | CH₃ | CH₃ | CH₂CH₃ | H |
| 061 | CH₃ | H | H | H | CH₂CH₃ | CH₃ |
| 062 | CH₃ | H | H | CH₃ | CH₂CH₃ | CH₃ |
| 063 | CH₃ | H | CH₃ | H | CH₂CH₃ | CH₃ |
| 064 | CH₃ | H | CH₃ | CH₃ | CH₂CH₃ | CH₃ |
| 065 | OCH₃ | H | H | H | CH₂CH₃ | H |
| 066 | OCH₃ | H | H | CH₃ | CH₂CH₃ | H |
| 067 | OCH₃ | H | CH₃ | H | CH₂CH₃ | H |
| 068 | OCH₃ | H | CH₃ | CH₃ | CH₂CH₃ | H |
| 069 | OCH₃ | H | H | H | CH₂CH₃ | CH₃ |
| 070 | OCH₃ | H | H | CH₃ | CH₂CH₃ | CH₃ |
| 071 | OCH₃ | H | CH₃ | H | CH₂CH₃ | CH₃ |
| 072 | OCH₃ | H | CH₃ | CH₃ | CH₂CH₃ | CH₃ |
| 073 | H | CH₃ | H | H | CH₂CH₃ | H |
| 074 | H | CH₃ | H | CH₃ | CH₂CH₃ | H |
| 075 | H | CH₃ | CH₃ | H | CH₂CH₃ | H |
| 076 | H | CH₃ | CH₃ | CH₃ | CH₂CH₃ | H |
| 077 | H | CH₃ | H | H | CH₂CH₃ | CH₃ |
| 078 | H | CH₃ | H | CH₃ | CH₂CH₃ | CH₃ |
| 079 | H | CH₃ | CH₃ | H | CH₂CH₃ | CH₃ |
| 080 | H | CH₃ | CH₃ | CH₃ | CH₂CH₃ | CH₃ |
| 081 | CH₃ | CH₃ | H | H | CH₂CH₃ | H |
| 082 | CH₃ | CH₃ | H | CH₃ | CH₂CH₃ | H |
| 083 | CH₃ | CH₃ | CH₃ | H | CH₂CH₃ | H |
| 084 | CH₃ | CH₃ | CH₃ | CH₃ | CH₂CH₃ | H |
| 085 | CH₃ | CH₃ | H | H | CH₂CH₃ | CH₃ |
| 086 | CH₃ | CH₃ | H | CH₃ | CH₂CH₃ | CH₃ |
| 087 | CH₃ | CH₃ | CH₃ | H | CH₂CH₃ | CH₃ |
| 088 | CH₃ | CH₃ | CH₃ | CH₃ | CH₂CH₃ | CH₃ |
| 089 | OCH₃ | CH₃ | H | H | CH₂CH₃ | H |
| 090 | OCH₃ | CH₃ | H | CH₃ | CH₂CH₃ | H |
| 091 | OCH₃ | CH₃ | CH₃ | H | CH₂CH₃ | H |
| 092 | OCH₃ | CH₃ | CH₃ | CH₃ | CH₂CH₃ | H |
| 093 | OCH₃ | CH₃ | H | H | CH₂CH₃ | CH₃ |
| 094 | OCH₃ | CH₃ | H | CH₃ | CH₂CH₃ | CH₃ |
| 095 | OCH₃ | CH₃ | CH₃ | H | CH₂CH₃ | CH₃ |
| 096 | OCH₃ | CH₃ | CH₃ | CH₃ | CH₂CH₃ | CH₃ |

Table A-1 provides 96 compounds A-1.001 to A-1.096 of formula (I) wherein R⁴ and R⁶ are both hydrogen, and RE, R², R³, R⁵, R⁷, and R⁸ are as defined in Table 1.
Table A-2 provides 96 compounds A-2.001 to A-2.096 of formula (I) wherein R⁴ is methyl, R⁶ is hydrogen, and R¹, R², R³, R⁵, R⁷, and R⁸ are as defined in Table 1.
Table A-3 provides 96 compounds A-3.001 to A-3.096 of formula (I) wherein R⁴ is ethyl, R⁶ is hydrogen, and R¹, R², R³, R⁵, R⁷, and R⁸ are as defined in Table 1.
Table A-4 provides 96 compounds A-4.001 to A-4.096 of formula (I) wherein R⁴ is methoxy, R⁶ is hydrogen, and R¹, R², R³, R⁵, R⁷, and R⁸ are as defined in Table 1.
Table A-5 provides 96 compounds A-5.001 to A-5.096 of formula (I) wherein R⁴ is methylsulfanyl, R⁶ is hydrogen, and R¹, R², R³, R⁵, R⁷, and R⁸ are as defined in Table 1.
Table A-6 provides 96 compounds A-6.001 to A-6.096 of formula (I) wherein R⁴ is methylsulfonyl, R⁶ is hydrogen, and R¹, R², R³, R⁵, R⁷, and R⁸ are as defined in Table 1.
Table A-7 provides 96 compounds A-7.001 to A-7.096 of formula (I) wherein R⁴ is hydrogen, R⁶ is methyl, and R¹, R², R³, R⁵, R⁷, and R⁸ are as defined in Table 1.
Table A-8 provides 96 compounds A-8.001 to A-8.096 of formula (I) wherein R⁴ and R⁶ are both methyl, and R¹, R², R³, R⁵, R⁷, and R⁸ are as defined in Table 1.
Table A-9 provides 96 compounds A-9.001 to A-9.096 of formula (I) wherein R⁴ is ethyl, R⁶ is methyl, and R¹, R², R³, R⁵, R⁷, and R⁸ are as defined in Table 1.
Table A-10 provides 96 compounds A-10.001 to A-10.096 of formula (I) wherein R⁴ is methoxy, R⁶ is methyl, and R¹, R², R³, R⁵, R⁷, and R⁸ are as defined in Table 1.
Table A-11 provides 96 compounds A-11.001 to A-11.096 of formula (I) wherein R⁴ is methylsulfanyl, R⁶ is methyl, and R¹, R², R³, R⁵, R⁷, and R⁸ are as defined in Table 1.
Table A-12 provides 96 compounds A-12.001 to A-12.096 of formula (I) wherein R⁴ is methylsulfonyl, R⁶ is methyl, and R¹, R², R³, R⁵, R⁷, and R⁸ are as defined in Table 1.
Table A-13 provides 96 compounds A-13.001 to A-13.096 of formula (I) wherein R⁴ is hydrogen, R⁶ is —CO₂CH₃, and R¹, R², R³, R⁵, R⁷, and R⁸ are as defined in Table 1.
Table A-14 provides 96 compounds A-14.001 to A-14.096 of formula (I) wherein R⁴ is methyl, R⁶ is —CO₂CH₃, and R¹, R², R³, R⁵, R⁷, and R⁸ are as defined in Table 1.
Table A-15 provides 96 compounds A-15.001 to A-15.096 of formula (I) wherein R⁴ is ethyl, R⁶ is —CO₂CH₃, and R¹, R², R³, R⁵, R⁷, and R⁸ are as defined in Table 1.
Table A-16 provides 96 compounds A-16.001 to A-16.096 of formula (I) wherein R⁴ is methoxy, R⁶ is —CO₂CH₃, and R¹, R², R³, R⁵, R⁷, and R⁸ are as defined in Table 1.
Table A-17 provides 96 compounds A-17.001 to A-17.096 of formula (I) wherein R⁴ is methylsulfanyl, R⁶ is —CO₂CH₃, and R¹, R², R³, R⁵, R⁷, and R⁸ are as defined in Table 1.
Table A-18 provides 96 compounds A-18.001 to A-18.096 of formula (I) wherein R⁴ is methylsulfonyl, R⁶ is —CO₂CH₃, and R¹, R², R³, R⁵, R⁷, and R⁸ are as defined in Table 1.
Table A-19 provides 96 compounds A-19.001 to A-19.096 of formula (I) wherein R⁴ is hydrogen, R⁶ is —CO₂N(CH₃)₂, and R¹, R², R³, R⁵, R⁷, and R⁸ are as defined in Table 1.
Table A-20 provides 96 compounds A-20.001 to A-20.096 of formula (I) wherein R⁴ is methyl, R⁶ is —CO₂N(CH₃)₂, and R¹, R², R³, R⁵, R⁷, and R⁸ are as defined in Table 1.
Table A-21 provides 96 compounds A-21.001 to A-21.096 of formula (I) wherein R⁴ is ethyl, R⁶ is —CO₂N(CH₃)₂, and R¹, R², R³, R⁵, R⁷, and R⁸ are as defined in Table 1.
Table A-22 provides 96 compounds A-22.001 to A-22.096 of formula (I) wherein R⁴ is methoxy, R⁶ is —CO₂N(CH₃)₂, and R¹, R², R³, R⁵, R⁷, and R⁸ are as defined in Table 1.
Table A-23 provides 96 compounds A-23.001 to A-23.096 of formula (I) wherein R⁴ is methylsulfanyl, R⁶ is —CO₂N(CH₃)₂, and R¹, R², R³, R⁵, R⁷, and R⁸ are as defined in Table 1.
Table A-24 provides 96 compounds A-24.001 to A-24.096 of formula (I) wherein R⁴ is methylsulfonyl, R⁶ is —CO₂N(CH₃)₂, and R¹, R², R³, R⁵, R⁷, and R⁸ are as defined in Table 1.
Table A-25 provides 96 compounds A-25.001 to A-25.096 of formula (I) wherein R⁴ is hydrogen, R⁶ is methylsulfonyl, and R¹, R², R³, R⁵, R⁷, and R⁸ are as defined in Table 1.
Table A-26 provides 96 compounds A-26.001 to A-26.096 of formula (I) wherein R⁴ is methyl, R⁶ is methylsulfonyl, and R¹, R², R³, R⁵, R⁷, and R⁸ are as defined in Table 1.
Table A-27 provides 96 compounds A-27.001 to A-27.096 of formula (I) wherein R⁴ is ethyl, R⁶ is methylsulfonyl, and R¹, R², R³, R⁵, R⁷, and R⁸ are as defined in Table 1.
Table A-28 provides 96 compounds A-28.001 to A-28.096 of formula (I) wherein R⁴ is methoxy, R⁶ is methylsulfonyl, and R¹, R², R³, R⁵, R⁷, and R⁸ are as defined in Table 1.
Table A-29 provides 96 compounds A-29.001 to A-29.096 of formula (I) wherein R⁴ is methylsulfanyl, R⁶ is methylsulfonyl, and R¹, R², R³, R⁵, R⁷, and R⁸ are as defined in Table 1.
Table A-30 provides 96 compounds A-30.001 to A-30.096 of formula (I) wherein R⁴ is methylsulfonyl, R⁶ is hydrogen, and R¹, R², R³, R⁵, R⁷, and R⁸ are as defined in Table 1.
Table A-31 provides 96 compounds A-31.001 to A-31.096 of formula (I) wherein R⁴ is hydrogen, R⁶ is methoxy, and R¹, R², R³, R⁵, R⁷, and R⁸ are as defined in Table 1.
Table A-32 provides 96 compounds A-32.001 to A-32.096 of formula (I) wherein R⁴ is methyl, R⁶ is methoxy, and R¹, R², R³, R⁵, R⁷, and R⁸ are as defined in Table 1.
Table A-33 provides 96 compounds A-33.001 to A-33.096 of formula (I) wherein R⁴ is ethyl, R⁶ is methoxy, and R¹, R², R³, R⁵, R⁷, and R⁸ are as defined in Table 1.
Table A-34 provides 96 compounds A-34.001 to A-34.096 of formula (I) wherein R⁴ is methoxy, R⁶ is methoxy, and R¹, R², R³, R⁵, R⁷, and R⁸ are as defined in Table 1.
Table A-35 provides 96 compounds A-35.001 to A-35.096 of formula (I) wherein R⁴ is methylsulfanyl, R⁶ is methoxy, and R¹, R², R³, R⁵, R⁷, and R⁸ are as defined in Table 1.
Table A-36 provides 96 compounds A-36.001 to A-36.096 of formula (I) wherein R⁴ is methylsulfonyl, R⁶ is methoxy, and R¹, R², R³, R⁵, R⁷, and R⁸ are as defined in Table 1.
Table A-37 provides 96 compounds A-37.001 to A-37.096 of formula (I) wherein R⁴ is hydrogen, R⁶ is cyano, and R¹, R², R³, R⁵, R⁷, and R³ are as defined in Table 1.
Table A-38 provides 96 compounds A-38.001 to A-38.096 of formula (I) wherein R⁴ is methyl, R⁶ is cyano, and R¹, R², R³, R⁵, R⁷, and R³ are as defined in Table 1.
Table A-39 provides 96 compounds A-39.001 to A-39.096 of formula (I) wherein R⁴ is ethyl, R⁶ is cyano, and R¹, R², R³, R⁵, R⁷, and R⁸ are as defined in Table 1.
Table A-40 provides 96 compounds A-40.001 to A-40.096 of formula (I) wherein R⁴ is methoxy, R⁶ is cyano, and R¹, R², R³, R⁵, R⁷, and R⁸ are as defined in Table 1.
Table A-41 provides 96 compounds A-41.001 to A-41.096 of formula (I) wherein R⁴ is methylsulfanyl, R⁶ is cyano, and R¹, R², R³, R⁵, R⁷, and R⁸ are as defined in Table 1.
Table A-42 provides 96 compounds A-42.001 to A-42.096 of formula (I) wherein R⁴ is methylsulfonyl, R⁶ is cyano, and R¹, R², R³, R⁵, R⁷, and R⁸ are as defined in Table 1.

Table B-1 provides 96 compounds B-1.001 to B-1.096 of formula (Ia) wherein and R¹, R², R³, R⁵, R⁷, and R⁸ are as defined in Table 1:

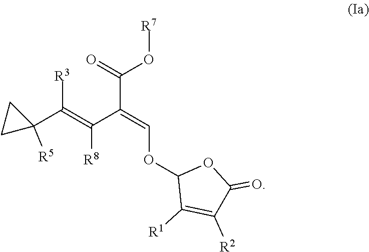

Table C-1 provides 96 compounds C-1.001 to C-1.096 of formula (Ib) wherein and R¹, R², R³, R⁵, R⁷, and R⁸ are as defined in Table 1:

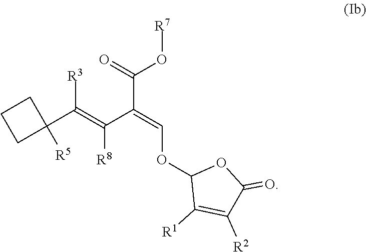

Table D-1 provides 96 compounds D-1.001 to D-1.096 of formula (Ic) wherein and R¹, R², R³, R⁵, R⁷, and R⁸ are as defined in Table 1:

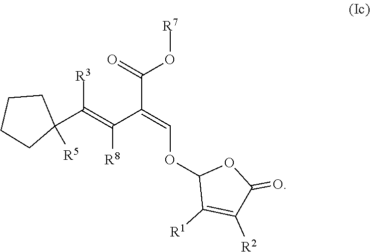

Table E-1 provides 96 compounds E-1.001 to E-1.096 of formula (Id) wherein and R¹, R², R³, R⁵, R⁷, and R⁸ are as defined in Table 1:

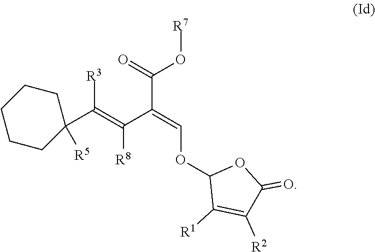

Table F-1 provides 96 compounds F-1.001 to F-1.096 of formula (Ie) wherein and R¹, R², R³, R⁵, R⁷, and R⁸ are as defined in Table 1:

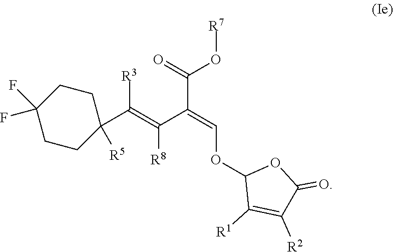

Table G-1 provides 96 compounds G-1.001 to G-1.096 of formula (If) wherein and R¹, R², R³, R⁷, and R⁸ are as defined in Table 1:

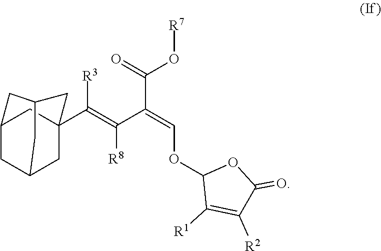

Table H-1 provides 96 compounds H-1.001 to H-1.096 of formula (Ig) wherein and R¹, R², R³, R⁵, R⁷, and R⁸ are as defined in Table 1:

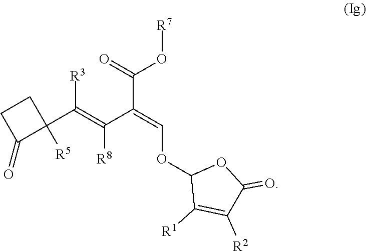

Table J-1 provides 96 compounds J-1.001 to J-1.096 of formula (Ih) wherein and R¹, R², R³, R⁷, and R⁸ are as defined in Table 1:

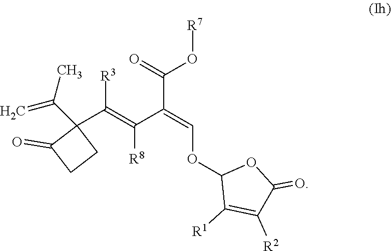

Table K-1 provides 96 compounds K-1.001 to K-1.096 of formula (Ii) wherein and R¹, R², R³, R⁵, R⁷, and R⁸ are as defined in Table 1:

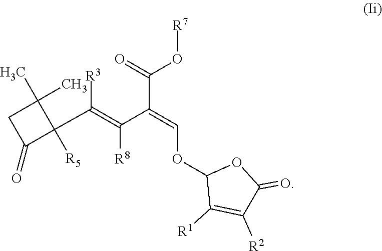

Table L-1 provides 96 compounds L-1.001 to L-1.096 of formula (Ij) wherein and R¹, R², R³, R⁵, R⁷, and R⁸ are as defined in Table 1:

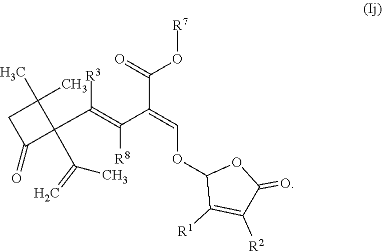

The present invention provides a method of improving the tolerance of a plant to abiotic stress, wherein the method comprises applying to the plant, plant part, plant propagation material, or plant growing locus a compound, composition, or mixture according to the present invention.

The present invention provides a method for regulating or improving the growth of a plant, wherein the method comprises applying to the plant, plant part, plant propagation material, or plant growing locus a compound, composition, or mixture according to the present invention. In one embodiment, plant growth is regulated or improved when the plant is subject to abiotic stress conditions.

The present invention also provides a method for improving seed germination of a plant, and especially the present invention provides a method for improving seed germination of a plant under cold stress conditions, comprising applying to the seed, or a locus containing seeds, a compound, a composition, or mixture according to the present invention.

The present invention also provides a method for safening a plant against phytotoxic effects of chemicals, comprising applying to the plant, plant part, plant propagation material, or plant growing locus a compound, a composition or mixture according to the present invention.

The present invention also provides a method for inducing/increasing leaf senescence in crops of useful plants, said method comprising applying to the plant, plant part, plant propagation material, or plant growing locus a compound, a composition or mixture according to the present invention. In particular, there is provided a method for inducing/increasing leaf senescence in corn, said method comprising applying to the corn plant, plant part, plant propagation material, or plant growing locus a compound, a composition or mixture according to the present invention.

According to the present invention, “regulating or improving the growth of a crop” means an improvement in plant vigour, an improvement in plant quality, improved tolerance to stress factors, and/or improved input use efficiency.

An ‘improvement in plant vigour’ means that certain traits are improved qualitatively or quantitatively when compared with the same trait in a control plant which has been grown under the same conditions in the absence of the method of the invention. Such traits include, but are not limited to, early and/or improved germination, improved emergence, the ability to use fewer seeds, increased root growth, a more developed root system, increased root nodulation, increased shoot growth, increased tillering, stronger tillers, more productive tillers, increased or improved plant stand, less plant verse (lodging), an increase and/or improvement in plant height, an increase in plant weight (fresh or dry), bigger leaf blades, greener leaf colour, increased pigment content, increased photosynthetic activity, earlier flowering, longer panicles, early grain maturity, increased seed, fruit or pod size, increased pod or ear number, increased seed number per pod or ear, increased seed mass, enhanced seed filling, fewer dead basal leaves, delay of senescence, improved vitality of the plant, increased levels of amino acids in storage tissues and/or fewer inputs needed (e.g. less fertiliser, water and/or labour needed). A plant with improved vigour may have an increase in any of the aforementioned traits or any combination or two or more of the aforementioned traits.

An ‘improvement in plant quality’ means that certain traits are improved qualitatively or quantitatively when compared with the same trait in a control plant which has been grown under the same conditions in the absence of the method of the invention. Such traits include, but are not limited to, improved visual appearance of the plant, reduced ethylene (reduced production and/or inhibition of reception), improved quality of harvested material, e.g. seeds, fruits, leaves, vegetables (such improved quality may manifest as improved visual appearance of the harvested material), improved carbohydrate content (e.g. increased quantities of sugar and/or starch, improved sugar acid ratio, reduction of reducing sugars, increased rate of development of sugar), improved protein content, improved oil content and composition, improved nutritional value, reduction in anti-nutritional compounds, improved organoleptic properties (e.g. improved taste) and/or improved consumer health benefits (e.g. increased levels of vitamins and anti-oxidants), improved post-harvest characteristics (e.g. enhanced shelf-life and/or storage stability, easier processability, easier extraction of compounds), more homogenous crop development (e.g. synchronised germination, flowering and/or fruiting of plants), and/or improved seed quality (e.g. for use in following seasons). A plant with improved quality may have an increase in any of the aforementioned traits or any combination or two or more of the aforementioned traits.

An ‘improved tolerance to stress factors’ means that certain traits are improved qualitatively or quantitatively when compared with the same trait in a control plant which has been grown under the same conditions in the absence of the method of the invention. Such traits include, but are not limited to, an increased tolerance and/or resistance to abiotic stress factors which cause sub-optimal growing conditions such as drought (e.g. any stress which leads to a lack of water content in plants, a lack of water uptake potential or a reduction in the water supply to plants), cold exposure, heat exposure, osmotic stress, UV stress, flooding, increased salinity (e.g. in the soil), increased mineral exposure, ozone exposure, high light exposure and/or limited availability of nutrients (e.g. nitrogen and/or phosphorus nutrients). A plant with improved tolerance to stress factors may have an increase in any of the aforementioned traits or any combination or two or more of the aforementioned traits. In the case of drought and nutrient stress, such improved tolerances may be due to, for example, more efficient uptake, use or retention of water and nutrients. In particular, the compounds or compositions of the present invention are useful to improve tolerance to drought stress.

An ‘improved input use efficiency’ means that the plants are able to grow more effectively using given levels of inputs compared to the growth of control plants which are grown under the same conditions in the absence of the method of the invention. In particular, the inputs include, but are not limited to fertiliser (such as nitrogen, phosphorous, potassium, and micronutrients), light and water. A plant with improved input use efficiency may have an improved use of any of the aforementioned inputs or any combination of two or more of the aforementioned inputs.

Other effects of regulating or improving the growth of a crop include a decrease in plant height, or reduction in tillering, which are beneficial features in crops or conditions where it is desirable to have less biomass and fewer tillers.

Any or all of the above crop enhancements may lead to an improved yield by improving e.g., plant physiology, plant growth and development and/or plant architecture. In the context of the present invention ‘yield’ includes, but is not limited to, (i) an increase in biomass production, grain yield, starch content, oil content and/or protein content, which may result from (a) an increase in the amount produced by the plant per se or (b) an improved ability to harvest plant matter, (ii) an improvement in the composition of the harvested material (e.g. improved sugar acid ratios, improved oil composition, increased nutritional value, reduction of anti-nutritional compounds, increased consumer health benefits) and/or (iii) an increased/facilitated ability to harvest the crop, improved processability of the crop and/or better storage stability/shelf life. Increased yield of an agricultural plant means that, where it is possible to take a quantitative measurement, the yield of a product of the respective plant is increased by a measurable amount over the yield of the same product of the plant produced under the same conditions, but without application of the present invention. According to the present invention, it is preferred that the yield be increased by at least 0.5%, more preferred at least 1%, even more preferred at least 2%, still more preferred at least 4%, preferably 5% or even more.

Any or all of the above crop enhancements may also lead to an improved utilisation of land, i.e., land which was previously unavailable or sub-optimal for cultivation may become available. For example, plants which show an increased ability to survive in drought conditions, may be able to be cultivated in areas of sub-optimal rainfall, e.g., perhaps on the fringe of a desert or even the desert itself.

In one aspect of the present invention, crop enhancements are made in the substantial absence of pressure from pests and/or diseases and/or abiotic stress. In a further aspect of the present invention, improvements in plant vigour, stress tolerance, quality and/or yield are made in the substantial absence of pressure from pests and/or diseases. For example, pests and/or diseases may be controlled by a pesticidal treatment that is applied prior to, or at the same time as, the method of the present invention. In a still further aspect of the present invention, improvements in plant vigour, stress tolerance, quality and/or yield are made in the absence of pest and/or disease pressure. In a further embodiment, improvements in plant vigour, quality and/or yield are made in the absence, or substantial absence, of abiotic stress.

The compound of formula (I) may be the sole active ingredient of a composition or it may be admixed with one or more additional active ingredients such as a pesticide, fungicide, synergist, herbicide, or plant growth regulator where appropriate. An additional active ingredient may, in some cases, result in unexpected synergistic activities.

Examples of suitable additional active ingredients also include the following: petroleum oils, 1,1-bis(4-chloro-phenyl)-2-ethoxyethanol, 2,4-dichlorophenyl benzenesulfonate, 2-fluoro-N-methyl-N-1-naphthylacetamide, 4-chlorophenyl phenyl sulfone, acetoprole, aldoxycarb, amidithion, amidothioate, amiton, amiton hydrogen oxalate, amitraz, aramite, arsenous oxide, azobenzene, azothoate, benomyl, benoxa-fos, benzyl benzoate, bixafen, brofenvalerate, bromo-cyclen, bromophos, bromopropylate, buprofezin, butocarboxim, butoxycarboxim, butylpyridaben, calcium polysulfide, camphechlor, carbanolate, carbophenothion, cymiazole, chino-methionat, chlorbenside, chlordimeform, chlordimeform hydrochloride, chlorfenethol, chlorfenson, chlorfensulfide, chlorobenzilate, chloromebuform, chloromethiuron, chloropropylate, chlorthiophos, cinerin I, cinerin II, cinerins, closantel, coumaphos, crotamiton, crotoxyphos, cufraneb, cyanthoate, DCPM, DDT, demephion, demephion-O, demephion-S, demeton-methyl, demeton-O, demeton-O-methyl, demeton-S, demeton-S-methyl, demeton-S-methylsulfon, dichlofluanid, dichlorvos, dicliphos, dienochlor, dimefox, dinex, dinex-diclexine, dinocap-4, dinocap-6, dinocton, dino-penton, dinosulfon, dinoterbon, dioxathion, diphenyl sulfone, disulfiram, DNOC, dofenapyn, doramectin, endothion, eprinomectin, ethoate-methyl, etrimfos, fenazaflor, fenbutatin oxide, fenothiocarb, fenpyrad, fen-pyroximate, fenpyrazamine, fenson, fentrifanil, flubenzimine, flucycloxuron, fluenetil, fluorbenside, FMC 1137, formetanate, formetanate hydrochloride, formparanate, gamma-HCH, glyodin, halfenprox, hexadecyl cyclopropanecarboxylate, isocarbophos, jasmolin I, jasmolin II, jodfenphos, lindane, malonoben, mecarbam, mephosfolan, mesulfen, methacrifos, methyl bromide, metolcarb, mexacarbate, milbemycin oxime, mipafox, monocrotophos, morphothion, moxidectin, naled, 4-chloro-2-(2-chloro-2-methyl-propyl)-5-[(6-iodo-3-pyridyl)methoxy]pyridazin-3-one, nifluridide, nikkomycins, nitrilacarb, nitrilacarb 1:1 zinc chloride complex, omethoate, oxydeprofos, oxydisulfoton, pp′-DDT, parathion, permethrin, phenkapton, phosalone, phosfolan, phosphamidon, polychloroterpenes, polynactins, proclonol, promacyl, propoxur, prothidathion, prothoate, pyrethrin I, pyrethrin II, pyrethrins, pyridaphenthion, pyrimitate, quinalphos, quintiofos, R-1492, phosglycin, rotenone, schradan, sebufos, selamectin, sophamide, SSI-121, sulfiram, sulfluramid, sulfotep, sulfur, diflovidazin, tau-fluvalinate, TEPP, terbam, tetradifon, tetrasul, thiafenox, thiocarboxime, thiofanox, thiometon, thioquinox, thuringiensin, triamiphos, triarathene, triazophos, triazuron, trifenofos, trinactin, vamidothion, vaniliprole, bethoxazin, copper dioctanoate, copper sulfate, cybutryne, dichlone, dichlorophen, endothal, fentin, hydrated lime, nabam, quinoclamine, quinonamid, simazine, triphenyltin acetate, triphenyltin hydroxide, crufomate, piperazine, thiophanate, chloralose, fenthion, pyridin-4-amine, strychnine, 1-hydroxy-1H-pyridine-2-thione, 4-(quinoxalin-2-ylamino)benzenesulfonamide, 8-hydroxyquinoline sulfate, bronopol, copper hydroxide, cresol, dipyrithione, dodicin, fenaminosulf, formaldehyde, hydrargaphen, kasugamycin, kasugamycin hydrochloride hydrate, nickel bis(dimethyldithiocarbamate), nitrapyrin, octhilinone, oxolinic acid, oxytetracycline, potassium hydroxyquinoline sulfate, probenazole, streptomycin, streptomycin sesquisulfate, tecloftalam, thiomersal, Adoxophyes orana GV, Agrobacterium radiobacter, Amblyseius spp., Anagrapha falcifera NPV, Anagrus atomus, Aphelinus abdominalis, Aphidius colemani, Aphidoletes aphidimyza, Autographa californica NPV, Bacillus sphaericus Neide, Beauveria brongniartii, Chrysoperla carnea, Cryptolaemus montrouzieri, Cydia pomonella GV, Dacnusa sibirica, Diglyphus isaea, Encarsia formosa, Eretmocerus eremicus, Heterorhabditis bacteriophora and H. megidis, Hippodamia convergens, Leptomastix dactylopii, Macrolophus caliginosus, Mamestra brassicae NPV, Metaphycus helvolus, Metarhizium anisopliae var. acridum, Metarhizium anisopliae var. anisopliae, Neodiprion sertifer NPV and N. lecontei NPV, Orius spp., Paecilomyces fumosoroseus, Phytoseiulus persimilis, Steinernema bibionis, Steinernema carpocapsae, Steinernema feltiae, Steinernema glaseri, Steinernema riobrave, Steinernema riobravis, Steinernema scapterisci, Steinernema spp., Trichogramma spp., Typhlodromus occidentalis, Verticillium lecanii, apholate, bisazir, busulfan, dimatif, hemel, hempa, metepa, methiotepa, methyl apholate, morzid, penfluron, tepa, thiohempa, thiotepa, tretamine, uredepa, (E)-dec-5-en-1-yl acetate with (E)-dec-5-en-1-ol, (E)-tridec-4-en-1-yl acetate, (E)-6-methylhept-2-en-4-ol, (E,Z)-tetradeca-4,10-dien-1-yl acetate, (Z)-dodec-7-en-1-yl acetate, (Z)-hexadec-11-enal, (Z)-hexadec-1-en-1-yl acetate, (Z)-hexadec-13-en-11-yn-1-yl acetate, (Z)-icos-13-en-10-one, (Z)-tetradec-7-en-1-al, (Z)-tetradec-9-en-1-ol, (Z)-tetradec-9-en-1-yl acetate, (7E,9Z)-dodeca-7,9-dien-1-yl acetate, (9Z,11E)-tetradeca-9,11-dien-1-yl acetate, (9Z,12E)-tetradeca-9,12-dien-1-yl acetate, 14-methyloctadec-1-ene, 4-methylnonan-5-ol with 4-methylnonan-5-one, alpha-multistriatin, brevicomin, codlelure, codlemone, cuelure, disparlure, dodec-8-en-1-yl acetate, dodec-9-en-1-yl acetate, dodeca-8,10-dien-1-yl acetate, dominicalure, ethyl 4-methyloctanoate, eugenol, frontalin, grandlure, grandlure I, grandlure II, grandlure III, grandlure IV, hexalure, ipsdienol, ipsenol, japonilure, lineatin, litlure, looplure, medlure, megatomoic acid, methyl eugenol, muscalure, octadeca-2,13-dien-1-yl acetate, octadeca-3,13-dien-1-yl acetate, orfralure, oryctalure, ostramone, siglure, sordidin, sulcatol, tetradec-11-en-1-yl acetate, trimedlure, trimedlure A, trimedlure B₁, trimedlure B₂, trimedlure C, trunc-call, 2-(octylthio)-ethanol, butopyronoxyl, butoxy(polypropylene glycol), dibutyl adipate, dibutyl phthalate, dibutyl succinate, diethyltoluamide, dimethyl carbate, dimethyl phthalate, ethyl hexanediol, hexamide, methoquin-butyl, methylneodecanamide, oxamate, picaridin, 1-dichloro-1-nitroethane, 1,1-dichloro-2,2-bis(4-ethylphenyl)-ethane, 1,2-dichloropropane with 1,3-dichloropropene, 1-bromo-2-chloroethane, 2,2,2-trichloro-1-(3,4-dichloro-phenyl)ethyl acetate, 2,2-dichlorovinyl 2-ethylsulfinylethyl methyl phosphate, 2-(1,3-dithiolan-2-yl)phenyl dimethylcarbamate, 2-(2-butoxyethoxy)ethyl thiocyanate, 2-(4,5-dimethyl-1,3-dioxolan-2-yl)phenyl methylcarbamate, 2-(4-chloro-3,5-xylyloxy)ethanol, 2-chlorovinyl diethyl phosphate, 2-imidazolidone, 2-isovalerylindan-1,3-dione, 2-methyl(prop-2-ynyl)aminophenyl methylcarbamate, 2-thiocyanatoethyl laurate, 3-bromo-1-chloroprop-1-ene, 3-methyl-1-phenylpyrazol-5-yl dimethyl-carbamate, 4-methyl(prop-2-ynyl)amino-3,5-xylyl methylcarbamate, 5,5-dimethyl-3-oxocyclohex-1-enyl dimethylcarbamate, acethion, acrylonitrile, aldrin, allosamidin, allyxycarb, alpha-ecdysone, aluminium phosphide, aminocarb, anabasine, athidathion, azamethiphos, Bacillus thuringiensis delta endotoxins, barium hexafluorosilicate, barium polysulfide, barthrin, Bayer 22/190, Bayer 22408, beta-cyfluthrin, beta-cypermethrin, bioethanomethrin, biopermethrin, bis(2-chloroethyl) ether, borax, bromfenvinfos, bromo-DDT, bufencarb, butacarb, butathiofos, butonate, calcium arsenate, calcium cyanide, carbon disulfide, carbon tetrachloride, cartap hydrochloride, cevadine, chlorbicyclen, chlordane, chlordecone, chloroform, chloropicrin, chlorphoxim, chlorprazophos, cis-resmethrin, cismethrin, clocythrin, copper acetoarsenite, copper arsenate, copper oleate, coumithoate, cryolite, CS 708, cyanofenphos, cyanophos, cyclethrin, cythioate, d-tetramethrin, DAEP, dazomet, decarbofuran, diamidafos, dicapthon, dichlofenthion, dicresyl, dicyclanil, dieldrin, diethyl 5-methylpyrazol-3-yl phosphate, dilor, dimefluthrin, dimetan, dimethrin, dimethylvinphos, dimetilan, dinoprop, dinosam, dinoseb, diofenolan, dioxabenzofos, dithicrofos, DSP, ecdysterone, EI 1642, EMPC, EPBP, etaphos, ethiofencarb, ethyl formate, ethylene dibromide, ethylene dichloride, ethylene oxide, EXD, fenchlorphos, fenethacarb, fenitrothion, fenoxacrim, fenpirithrin, fensulfothion, fenthion-ethyl, flucofuron, fosmethilan, fospirate, fosthietan, furathiocarb, furethrin, guazatine, guazatine acetates, sodium tetrathiocarbonate, halfenprox, HCH, HEOD, heptachlor, heterophos, HHDN, hydrogen cyanide, hyquincarb, IPSP, isazofos, isobenzan, isodrin, isofenphos, isolane, isoprothiolane, isoxathion, juvenile hormone I, juvenile hormone II, juvenile hormone III, kelevan, kinoprene, lead arsenate, leptophos, lirimfos, lythidathion, m-cumenyl methylcarbamate, magnesium phosphide, mazidox, mecarphon, menazon, mercurous chloride, mesulfenfos, metam, metam-potassium, metam-sodium, methanesulfonyl fluoride, methocrotophos, methoprene, methothrin, methoxychlor, methyl isothiocyanate, methylchloroform, methylene chloride, metoxadiazone, mirex, naftalofos, naphthalene, NC-170, nicotine, nicotine sulfate, nithiazine, nornicotine, O-5-dichloro-4-iodophenyl O-ethyl ethylphosphonothioate, O,O-diethyl O-4-methyl-2-oxo-2H-chromen-7-yl phosphorothioate, O,O-diethyl O-6-methyl-2-propylpyrimidin-4-yl phosphorothioate, O,O,O′,O′-tetrapropyl dithiopyrophosphate, oleic acid, para-dichlorobenzene, parathion-methyl, pentachlorophenol, pentachlorophenyl laurate, PH 60-38, phenkapton, phosnichlor, phosphine, phoxim-methyl, pirimetaphos, polychlorodicyclopentadiene isomers, potassium arsenite, potassium thiocyanate, precocene I, precocene II, precocene III, primidophos, profluthrin, promecarb, prothiofos, pyrazophos, pyresmethrin, quassia, quinalphos-methyl, quinothion, rafoxanide, resmethrin, rotenone, kadethrin, ryania, ryanodine, sabadilla, schradan, sebufos, SI-0009, thiapronil, sodium arsenite, sodium cyanide, sodium fluoride, sodium hexafluorosilicate, sodium pentachlorophenoxide, sodium selenate, sodium thiocyanate, sulcofuron, sulcofuron-sodium, sulfuryl fluoride, sulprofos, tar oils, tazimcarb, TDE, tebupirimfos, temephos, terallethrin, tetrachloroethane, thicrofos, thiocyclam, thiocyclam hydrogen oxalate, thionazin, thiosultap, thiosultap-sodium, tralomethrin, transpermethrin, triazamate, trichlormetaphos-3, trichloronat, trimethacarb, tolprocarb, triclopyricarb, triprene, veratridine, veratrine, XMC, zetamethrin, zinc phosphide, zolaprofos, meperfluthrin, tetramethylfluthrin, bis(tributyltin) oxide, bromoacetamide, ferric phosphate, niclosamide-olamine, tributyltin oxide, pyrimorph, trifenmorph, 1,2-dibromo-3-chloropropane, 1,3-dichloropropene, 3,4-dichlorotetrahydrothio-phene 1,1-dioxide, 3-(4-chlorophenyl)-5-methylrhodanine, 5-methyl-6-thioxo-1,3,5-thiadiazinan-3-ylacetic acid, 6-isopentenylaminopurine, anisiflupurin, benclothiaz, cytokinins, DCIP, furfural, isamidofos, kinetin, Myrothecium verrucaria composition, tetrachlorothiophene, xylenols, zeatin, potassium ethylxanthate, acibenzolar, acibenzolar-S-methyl, Reynoutria sachalinensis extract, alpha-chlorohydrin, antu, barium carbonate, bisthiosemi, brodifacoum, bromadiolone, bromethalin, chlorophacinone, cholecalciferol, coumachlor, coumafuryl, coumatetralyl, crimidine, difenacoum, difethialone, diphacinone, ergocalciferol, flocoumafen, fluoroacetamide, flupropadine, flupropadine hydrochloride, norbormide, phosacetim, phosphorus, pindone, pyrinuron, scilliroside, sodium fluoro-acetate, thallium sulfate, warfarin, 2-(2-butoxyethoxy)-ethyl piperonylate, 5-(1,3-benzodioxol-5-yl)-3-hexylcyclohex-2-enone, farnesol with nerolidol, verbutin, MGK 264, piperonyl butoxide, piprotal, propyl isomer, S421, sesamex, sesasmolin, sulfoxide, anthraquinone, copper naphthenate, copper oxychloride, dicyclopentadiene, thiram, zinc naphthenate, ziram, imanin, ribavirin, chloroinconazide, mercuric oxide, thiophanate-methyl, azaconazole, bitertanol, bromuconazole, cyproconazole, difenoconazole, diniconazole, epoxicon-azole, fenbuconazole, fluquinconazole, flusilazole, flutriafol, furametpyr, hexaconazole, imazalil, imiben-con-azole, ipconazole, metconazole, myclobutanil, paclobutrazole, pefurazoate, penconazole, prothioconazole, pyrifenox, prochloraz, propiconazole, pyrisoxazole, simeconazole, tebucon-azole, tetraconazole, triadimefon, triadime-nol, triflumizole, triticonazole, ancymidol, fenarimol, nuarimol, bupirimate, dimethirimol, ethirimol, dodemorph, fenpropidin, fenpropimorph, spiroxamine, tridemorph, cyprodinil, mepanipyrim, pyrimethanil, fenpiclonil, fludioxonil, benalaxyl, furalaxyl, meta-laxyl, R-metalaxyl, ofurace, oxadixyl, carbendazim, debacarb, fuberidazole, thiaben-dazole, chlozolinate, dichlozoline, myclozoline, procymi-done, vinclozoline, boscalid, carboxin, fenfuram, flutolanil, mepronil, oxycarboxin, penthiopyrad, thifluzamide, dodine, iminoctadine, azoxystrobin, dimoxystrobin, enestroburin, fenaminstrobin, flufenoxystrobin, fluoxastrobin, kresoxim-methyl, metomi-nostrobin, trifloxystrobin, orysastrobin, picoxystrobin, pyraclostrobin, pyrametostrobin, pyraoxystrobin, ferbam, mancozeb, maneb, metiram, propineb, zineb, captafol, captan, fluoroimide, folpet, tolylfluanid, bordeaux mixture, copper oxide, mancopper, oxine-copper, nitrothal-isopropyl, edifenphos, iprobenphos, phosdiphen, tolclofos-methyl, anilazine, benthiavalicarb, blasticidin-S, chloroneb, chloro-tha-lo-nil, cyflufenamid, cymoxanil, cyclobutrifluram, diclocymet, diclomezine, dicloran, diethofencarb, dimetho-morph, flumorph, dithianon, ethaboxam, etridiazole, famoxa-done, fenamidone, fenoxanil, ferimzone, fluazinam, flumetylsulforim, fluopicolide, fluoxytioconazole, flusulfamide, fluxapyroxad, fenhexamid, fos-etyl-aluminium, hymexazol, iprovalicarb, cyazofamid, methasulfo-carb, metrafenone, pencycuron, phthalide, polyoxins, propamocarb, pyribencarb, proquinazid, pyroquilon, pyriofenone, quinoxyfen, quintozene, tiadinil, triazoxide, tricyclazole, triforine, validamycin, valifenalate, zoxamide, mandipropamid, flubeneteram, isopyrazam, sedaxane, benzovindiflupyr, pydiflumetofen, 3-difluoromethyl-1-methyl-1H-pyrazole-4-carboxylic acid (3′,4′,5′-trifluoro-biphenyl-2-yl)-amide, isoflucypram, isotianil, dipymetitrone, 6-ethyl-5,7-dioxo-pyrrolo[4,5][1,4]dithiino[1,2-c]isothiazole-3-carbonitrile, 2-(difluoromethyl)-N-[3-ethyl-1,1-dimethyl-indan-4-yl]pyridine-3-carboxamide, 4-(2,6-difluorophenyl)-6-methyl-5-phenyl-pyridazine-3-carbonitrile, (R)-3-(difluoromethyl)-1-methyl-N-[1,1,3-trimethylindan-4-yl]pyrazole-4-carboxamide, 4-(2-bromo-4-fluoro-phenyl)-N-(2-chloro-6-fluoro-phenyl)-2,5-dimethyl-pyrazol-3-amine, 4-(2-bromo-4-fluorophenyl)-N-(2-chloro-6-fluorophenyl)-1,3-dimethyl-1H-pyrazol-5-amine, fluindapyr, coumethoxystrobin (jiaxiangjunzhi), Ivbenmixianan, dichlobentiazox, mandestrobin, 3-(4,4-difluoro-3,4-dihydro-3,3-dimethylisoquinolin-1-yl)quinolone, 2-[2-fluoro-6-[(8-fluoro-2-methyl-3-quinolyl)oxy]phenyl]propan-2-ol, oxathiapiprolin, tert-butyl N-[6-[[[(1-methyltetrazol-5-yl)-phenyl-methylene]amino]oxymethyl]-2-pyridyl]carbamate, pyraziflumid, inpyrfluxam, trolprocarb, mefentrifluconazole, ipfentrifluconazole, 2-(difluoromethyl)-N-[(3R)-3-ethyl-1,1-dimethyl-indan-4-yl]pyridine-3-carboxamide, N′-(2,5-dimethyl-4-phenoxy-phenyl)-N-ethyl-N-methyl-formamidine, N′-[4-(4,5-dichlorothiazol-2-yl)oxy-2,5-dimethylphenyl]-N-ethyl-N-methyl-formamidine, [2-[3-[2-[1-[2-[3,5-bis(difluoromethyl)pyrazol-1-yl]acetyl]-4-piperidyl]thiazol-4-yl]-4,5-dihydroisoxazol-5-yl]-3-chloro-phenyl]methanesulfonate, but-3-ynyl N-[6-[[(Z)-[(1-methyltetrazol-5-yl)-phenyl-methylene]amino]oxymethyl]-2-pyridyl]carbamate, methyl N-[[5-[4-(2,4-dimethylphenyl)triazol-2-yl]-2-methyl-phenyl]methyl]carbamate, 3-chloro-6-methyl-5-phenyl-4-(2,4,6-trifluorophenyl)pyridazine, pyridachlometyl, 3-(difluoromethyl)-1-methyl-N-[1,1,3-trimethylindan-4-yl]pyrazole-4-carboxamide, 1-[2-[[1-(4-chlorophenyl)pyrazol-3-yl]oxymethyl]-3-methyl-phenyl]-4-methyl-tetrazol-5-one, 1-methyl-4-[3-methyl-2-[[2-methyl-4-(3,4,5-trimethylpyrazol-1-yl)phenoxy]methyl]phenyl]tetrazol-5-one, aminopyrifen, ametoctradin, amisulbrom, penflufen, (Z,2E)-5-[1-(4-chlorophenyl)pyrazol-3-yl]oxy-2-methoxyimino-N,3-dimethyl-pent-3-enamide, florylpicoxamid, fenpicoxamid, metarylpicoxamid, tebufloquin, ipflufenoquin, quinofumelin, isofetamid, N-[2-[2,4-dichloro-phenoxy]phenyl]-3-(difluoromethyl)-1-methyl-pyrazole-4-carboxamide, N-[2-[2-chloro-4-(trifluoromethyl)phenoxy]phenyl]-3-(difluoromethyl)-1-methyl-pyrazole-4-carboxamide, benzothiostrobin, phenamacril, 5-amino-1,3,4-thiadiazole-2-thiol zinc salt (2:1), fluopyram, flufenoxadiazam, flutianil, fluopimomide, pyrapropoyne, picarbutrazox, 2-(difluoromethyl)-N-(3-ethyl-1,1-dimethyl-indan-4-yl)pyridine-3-carboxamide, 2-(difluoromethyl)-N-((3R)-1,1,3-trimethylindan-4-yl) pyridine-3-carboxamide, 4-[[6-[2-(2,4-difluorophenyl)-1,1-difluoro-2-hydroxy-3-(1,2,4-triazol-1-yl)propyl]-3-pyridyl]oxy]benzonitrile, metyltetraprole, 2-(difluoromethyl)-N-((3R)-1,1,3-trimethylindan-4-yl) pyridine-3-carboxamide, α-(1,1-dimethylethyl)-α-[4′-(trifluoromethoxy) [1,1′-biphenyl]-4-yl]-5-pyrimidinemethanol, fluoxapiprolin, enoxastrobin, 4-[[6-[2-(2,4-difluorophenyl)-1,1-difluoro-2-hydroxy-3-(1,2,4-triazol-1-yl)propyl]-3-pyridyl]oxy]benzonitrile, 4-[[6-[2-(2,4-difluorophenyl)-1,1-difluoro-2-hydroxy-3-(5-sulfanyl-1,2,4-triazol-1-yl)propyl]-3-pyridyl]oxy]benzonitrile, 4-[[6-[2-(2,4-difluorophenyl)-1,1-difluoro-2-hydroxy-3-(5-thioxo-4H-1,2,4-triazol-1-yl)propyl]-3-pyridyl]oxy]benzonitrile, trinexapac, coumoxystrobin, zhongshengmycin, thiodiazole copper, zinc thiazole, amectotractin, iprodione, seboctylamine, N′-[5-bromo-2-methyl-6-[(1S)-1-methyl-2-propoxy-ethoxy]-3-pyridyl]-N-ethyl-N-methyl-formamidine, N′-[5-bromo-2-methyl-6-[(1R)-1-methyl-2-propoxy-ethoxy]-3-pyridyl]-N-ethyl-N-methyl-formamidine, N′-[5-bromo-2-methyl-6-(1-methyl-2-propoxy-ethoxy)-3-pyridyl]-N-ethyl-N-methyl-formamidine, N′-[5-chloro-2-methyl-6-(1-methyl-2-propoxy-ethoxy)-3-pyridyl]-N-ethyl-N-methyl-formamidine, N′-[5-bromo-2-methyl-6-(1-methyl-2-propoxy-ethoxy)-3-pyridyl]-N-isopropyl-N-methyl-formamidine (these compounds may be prepared from the methods described in WO2015/155075); N′-[5-bromo-2-methyl-6-(2-propoxypropoxy)-3-pyridyl]-N-ethyl-N-methyl-formamidine (this compound may be prepared from the methods described in IPCOM000249876D); N-isopropyl-N′-[5-methoxy-2-methyl-4-(2,2,2-trifluoro-1-hydroxy-1-phenyl-ethyl)phenyl]-N-methyl-formamidine, N′-[4-(1-cyclopropyl-2,2,2-trifluoro-1-hydroxy-ethyl)-5-methoxy-2-methyl-phenyl]-N-isopropyl-N-methyl-formamidine (these compounds may be prepared from the methods described in WO2018/228896); N-ethyl-N′-[5-methoxy-2-methyl-4-[(2-trifluoromethyl)oxetan-2-yl]phenyl]-N-methyl-formamidine, N-ethyl-N′-[5-methoxy-2-methyl-4-[(2-trifuoromethyl)tetrahydrofuran-2-yl]phenyl]-N-methyl-formamidine (these compounds may be prepared from the methods described in WO2019/110427); N-[(1R)-1-benzyl-3-chloro-1-methyl-but-3-enyl]-8-fluoro-quinoline-3-carboxamide, N-[(1S)-1-benzyl-3-chloro-1-methyl-but-3-enyl]-8-fluoro-quinoline-3-carboxamide, N-[(1R)-1-benzyl-3,3,3-trifluoro-1-methyl-propyl]-8-fluoro-quinoline-3-carboxamide, N-[(1S)-1-benzyl-3,3,3-trifluoro-1-methyl-propyl]-8-fluoro-quinoline-3-carboxamide, N-[(1R)-1-benzyl-1,3-dimethyl-butyl]-7,8-difluoro-quinoline-3-carboxamide, N-[(1S)-1-benzyl-1,3-dimethyl-butyl]-7,8-difluoro-quinoline-3-carboxamide, 8-fluoro-N-[(1R)-1-[(3-fluorophenyl)methyl]-1,3-dimethyl-butyl]quinoline-3-carboxamide, 8-fluoro-N-[(1S)-1-[(3-fluorophenyl)methyl]-1,3-dimethyl-butyl]quinoline-3-carboxamide, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, N-((1R)-1-benzyl-3-chloro-1-methyl-but-3-enyl)-8-fluoro-quinoline-3-carboxamide, N-((1S)-1-benzyl-3-chloro-1-methyl-but-3-enyl)-8-fluoro-quinoline-3-carboxamide (these compounds may be prepared from the methods described in WO2017/153380); 1-(6,7-dimethylpyrazolo[1,5-a]pyridin-3-yl)-4,4,5-trifluoro-3,3-dimethyl-isoquinoline, 1-(6,7-dimethylpyrazolo[1,5-a]pyridin-3-yl)-4,4,6-trifluoro-3,3-dimethyl-isoquinoline, 4,4-difluoro-3,3-dimethyl-1-(6-methylpyrazolo[1,5-a]pyridin-3-yl)isoquinoline, 4,4-difluoro-3,3-dimethyl-1-(7-methylpyrazolo[1,5-a]pyridin-3-yl)isoquinoline, 1-(6-chloro-7-methylpyrazolo[1,5-a]pyridin-3-yl)-4,4-difluoro-3,3-dimethyl-isoquinoline (these compounds may be prepared from the methods described in WO2017/025510); 1-(4,5-dimethylbenzimidazol-1-yl)-4,4,5-trifluoro-3,3-dimethyl-isoquinoline, 1-(4,5-dimethylbenzimidazol-1-yl)-4,4-difluoro-3,3-dimethyl-isoquinoline, 6-chloro-4,4-difluoro-3,3-dimethyl-1-(4-methylbenzimidazol-1-yl)isoquinoline, 4,4-difluoro-1-(5-fluoro-4-methyl-benzimidazol-1-yl)-3,3-dimethyl-isoquinoline, 3-(4,4-difluoro-3,3-dimethyl-1-isoquinolyl)-7,8-dihydro-6H-cyclopenta[e]benzimidazole (these compounds may be prepared from the methods described in WO2016/156085); N-methoxy-N-[[4-[5-(trifluoromethyl)-1,2,4-oxadiazol-3-yl]phenyl]methyl]cyclopropanecarboxamide, N,2-dimethoxy-N-[[4-[5-(trifluoromethyl)-1,2,4-oxadiazol-3-yl]phenyl]methyl]propanamide, N-ethyl-2-methyl-N-[[4-[5-(trifluoromethyl)-1,2,4-oxadiazol-3-yl]phenyl]methyl]propanamide, 1-methoxy-3-methyl-1-[[4-[5-(trifluoromethyl)-1,2,4-oxadiazol-3-yl]phenyl]methyl]urea, 1,3-dimethoxy-1-[[4-[5-(trifluoromethyl)-1,2,4-oxadiazol-3-yl]phenyl]methyl]urea, 3-ethyl-1-methoxy-1-[[4-[5-(trifluoromethyl)-1,2,4-oxadiazol-3-yl]phenyl]methyl]urea, N-[[4-[5-(trifluoromethyl)-1,2,4-oxadiazol-3-yl]phenyl]methyl]propanamide, 4,4-dimethyl-2-[[4-[5-(trifluoromethyl)-1,2,4-oxadiazol-3-yl]phenyl]methyl]isoxazolidin-3-one, 5,5-dimethyl-2-[[4-[5-(trifluoromethyl)-1,2,4-oxadiazol-3-yl]phenyl]methyl]isoxazolidin-3-one, ethyl 1-[[4-[5-(trifluoromethyl)-1,2,4-oxadiazol-3-yl]phenyl]methyl]pyrazole-4-carboxylate, N,N-dimethyl-1-[[4-[5-(trifluoromethyl)-1,2,4-oxadiazol-3-yl]phenyl]methyl]-1,2,4-triazol-3-amine (these compounds may be prepared from the methods described in WO 2017/055473, WO 2017/055469, WO 2017/093348 and WO 2017/118689); 2-[6-(4-chlorophenoxy)-2-(trifluoromethyl)-3-pyridyl]-1-(1,2,4-triazol-1-yl)propan-2-ol (this compound may be prepared from the methods described in WO 2017/029179); 2-[6-(4-bromophenoxy)-2-(trifluoromethyl)-3-pyridyl]-1-(1,2,4-triazol-1-yl)propan-2-ol (this compound may be prepared from the methods described in WO 2017/029179); 3-[2-(1-chlorocyclopropyl)-3-(2-fluorophenyl)-2-hydroxy-propyl]imidazole-4-carbonitrile (this compound may be prepared from the methods described in WO 2016/156290); 3-[2-(1-chlorocyclopropyl)-3-(3-chloro-2-fluoro-phenyl)-2-hydroxy-propyl]imidazole-4-carbonitrile (this compound may be prepared from the methods described in WO 2016/156290); (4-phenoxyphenyl)methyl 2-amino-6-methyl-pyridine-3-carboxylate (this compound may be prepared from the methods described in WO 2014/006945); 2,6-Dimethyl-1H,5H-[1,4]dithiino[2,3-c:5,6-c′]dipyrrole-1,3,5,7(2H,6H)-tetrone (this compound may be prepared from the methods described in WO 2011/138281) N-methyl-4-[5-(trifluoromethyl)-1,2,4-oxadiazol-3-yl]benzenecarbothioamide; N-methyl-4-[5-(trifluoromethyl)-1,2,4-oxadiazol-3-yl]benzamide; (Z,2E)-5-[1-(2,4-dichlorophenyl)pyrazol-3-yl]oxy-2-methoxyimino-N,3-dimethyl-pent-3-enamide (this compound may be prepared from the methods described in WO 2018/153707); N′-(2-chloro-5-methyl-4-phenoxy-phenyl)-N-ethyl-N-methyl-formamidine; N′-[2-chloro-4-(2-fluorophenoxy)-5-methyl-phenyl]-N-ethyl-N-methyl-formamidine (this compound may be prepared from the methods described in WO 2016/202742); 2-(difluoromethyl)-N-[(3S)-3-ethyl-1,1-dimethyl-indan-4-yl]pyridine-3-carboxamide (this compound may be prepared from the methods described in WO 2014/095675); (5-methyl-2-pyridyl)-[4-[5-(trifluoromethyl)-1,2,4-oxadiazol-3-yl]phenyl]methanone, (3-methylisoxazol-5-yl)-[4-[5-(trifluoromethyl)-1,2,4-oxadiazol-3-yl]phenyl]methanone (these compounds may be prepared from the methods described in WO 2017/220485); 2-oxo-N-propyl-2-[4-[5-(trifluoromethyl)-1,2,4-oxadiazol-3-yl]phenyl]acetamide (this compound may be prepared from the methods described in WO 2018/065414); ethyl 1-[[5-[5-(trifluoromethyl)-1,2,4-oxadiazol-3-yl]-2-thienyl]methyl]pyrazole-4-carboxylate (this compound may be prepared from the methods described in WO 2018/158365); 2,2-difluoro-N-methyl-2-[4-[5-(trifluoromethyl)-1,2,4-oxadiazol-3-yl]phenyl]acetamide, N—[(E)-methoxyiminomethyl]-4-[5-(trifluoromethyl)-1,2,4-oxadiazol-3-yl]benzamide, N—[(Z)-methoxyiminomethyl]-4-[5-(trifluoromethyl)-1,2,4-oxadiazol-3-yl]benzamide, N—[N-methoxy-C-methyl-carbonimidoyl]-4-[5-(trifluoromethyl)-1,2,4-oxadiazol-3-yl]benzamide (these compounds may be prepared from the methods described in WO 2018/202428).

The compounds of the present invention can be used alone, but are generally formulated into compositions using formulation adjuvants, such as carriers, solvents, and surface-active agents (SFAs). Thus, the present invention further provides a composition comprising a compound of the present invention and an agriculturally acceptable formulation adjuvant. There is also provided a composition consisting essentially of a compound of the present invention and an agriculturally acceptable formulation adjuvant. There is also provided a composition consisting of a compound of the present invention and an agriculturally acceptable formulation adjuvant.

The present invention further provides a plant growth regulator composition comprising a compound of the present invention and an agriculturally acceptable formulation adjuvant. There is also provided a plant growth regulator composition consisting essentially of a compound of the present invention and an agriculturally acceptable formulation adjuvant. There is also provided a plant growth regulator composition consisting of a compound of the present invention and an agriculturally acceptable formulation adjuvant.

The present invention further provides a plant abiotic stress management composition comprising a compound of the present invention and an agriculturally acceptable formulation adjuvant. There is also provided a plant abiotic stress management composition consisting essentially of a compound of the present invention and an agriculturally acceptable formulation adjuvant. There is also provided a plant abiotic stress management composition consisting of a compound of the present invention and an agriculturally acceptable formulation adjuvant.

The present invention further provides a seed germination promoter composition comprising a compound of the present invention and an agriculturally acceptable formulation adjuvant. There is also provided a seed germination promoter composition consisting essentially of a compound of the present invention and an agriculturally acceptable formulation adjuvant. There is also provided a seed germination promoter composition consisting of a compound of the present invention and an agriculturally acceptable formulation adjuvant.

The composition can be in the form of concentrates which are diluted prior to use, although ready-to-use compositions can also be made. The final dilution is usually made with water, but can be made instead of, or in addition to, water, with, for example, liquid fertilisers, micronutrients, biological organisms, oil, or solvents.

The compositions generally comprise from 0.1 to 99% by weight, especially from 0.1 to 95% by weight, compounds of the present invention are from 1 to 99.9% by weight of a formulation adjuvant which preferably includes from 0 to 25% by weight of a surface-active substance.

The compositions can be chosen from a number of formulation types, many of which are known from the Manual on Development and Use of FAO Specifications for Plant Protection Products, 5th Edition, 1999. These include dustable powders (DP), soluble powders (SP), water soluble granules (SG), water dispersible granules (WG), wettable powders (WP), granules (GR) (slow or fast release), soluble concentrates (SL), oil miscible liquids (OL), ultralow volume liquids (UL), emulsifiable concentrates (EC), dispersible concentrates (DC), emulsions (both oil in water (EW) and water in oil (EO)), micro-emulsions (ME), suspension concentrates (SC), aerosols, capsule suspensions (CS) and seed treatment formulations. The formulation type chosen in any instance will depend upon the particular purpose envisaged and the physical, chemical, and biological properties of the compound of the present invention.

Dustable powders (DP) may be prepared by mixing a compound of the present invention with one or more solid diluents (for example natural clays, kaolin, pyrophyllite, bentonite, alumina, montmorillonite, kieselguhr, chalk, diatomaceous earths, calcium phosphates, calcium and magnesium carbonates, sulfur, lime, flours, talc and other organic and inorganic solid carriers) and mechanically grinding the mixture to a fine powder.

Soluble powders (SP) may be prepared by mixing a compound of the present invention with one or more water-soluble inorganic salts (such as sodium bicarbonate, sodium carbonate or magnesium sulphate) or one or more water-soluble organic solids (such as a polysaccharide) and, optionally, one or more wetting agents, one or more dispersing agents or a mixture of said agents to improve water dispersibility/solubility. The mixture is then ground to a fine powder. Similar compositions may also be granulated to form water soluble granules (SG).

Wettable powders (WP) may be prepared by mixing a compound of the present invention with one or more solid diluents or carriers, one or more wetting agents and, preferably, one or more dispersing agents and, optionally, one or more suspending agents to facilitate the dispersion in liquids. The mixture is then ground to a fine powder. Similar compositions may also be granulated to form water dispersible granules (WG).

Granules (GR) may be formed either by granulating a mixture of a compound of the present invention and one or more powdered solid diluents or carriers, or from pre-formed blank granules by absorbing a compound of the present invention (or a solution thereof, in a suitable agent) in a porous granular material (such as pumice, attapulgite clays, fuller's earth, kieselguhr, diatomaceous earths or ground corn cobs) or by adsorbing a compound of the present invention (or a solution thereof, in a suitable agent) on to a hard core material (such as sands, silicates, mineral carbonates, sulphates or phosphates) and drying if necessary. Agents which are commonly used to aid absorption or adsorption include solvents (such as aliphatic and aromatic petroleum solvents, alcohols, ethers, ketones and esters) and sticking agents (such as polyvinyl acetates, polyvinyl alcohols, dextrins, sugars and vegetable oils). One or more other additives may also be included in granules (for example an emulsifying agent, wetting agent or dispersing agent).

Dispersible Concentrates (DC) may be prepared by dissolving a compound of the present invention in water or an organic solvent, such as a ketone, alcohol or glycol ether. These solutions may contain a surface-active agent (for example to improve water dilution or prevent crystallisation in a spray tank).

Emulsifiable concentrates (EC) or oil-in-water emulsions (EW) may be prepared by dissolving a compound of the present invention in an organic solvent (optionally containing one or more wetting agents, one or more emulsifying agents or a mixture of said agents). Suitable organic solvents for use in ECs include aromatic hydrocarbons (such as alkylbenzenes or alkylnaphthalenes, exemplified by SOLVESSO 100, SOLVESSO 150 and SOLVESSO 200; SOLVESSO is a Registered Trade Mark), ketones (such as cyclohexanone or methylcyclohexanone) and alcohols (such as benzyl alcohol, furfuryl alcohol or butanol), N-alkylpyrrolidones (such as N-methylpyrrolidone or N-octylpyrrolidone), dimethyl amides of fatty acids (such as C₈-C₁₀ fatty acid dimethylamide) and chlorinated hydrocarbons. An EC product may spontaneously emulsify on addition to water, to produce an emulsion with sufficient stability to allow spray application through appropriate equipment.

Preparation of an EW involves obtaining a compound of the present invention either as a liquid (if it is not a liquid at room temperature, it may be melted at a reasonable temperature, typically below 70° C.) or in solution (by dissolving it in an appropriate solvent) and then emulsifying the resultant liquid or solution into water containing one or more SFAs, under high shear, to produce an emulsion. Suitable solvents for use in EWs include vegetable oils, chlorinated hydrocarbons (such as chlorobenzenes), aromatic solvents (such as alkylbenzenes or alkylnaphthalenes) and other appropriate organic solvents which have a low solubility in water.

Microemulsions (ME) may be prepared by mixing water with a blend of one or more solvents with one or more SFAs, to produce spontaneously a thermodynamically stable isotropic liquid formulation. A compound of the present invention is present initially in eitherthe water orthe solvent/SFA blend. Suitable solvents for use in MEs include those hereinbefore described for use in ECs or in EWs. An ME may be either an oil-in-water or a water-in-oil system (which system is present may be determined by conductivity measurements) and may be suitable for mixing water-soluble and oil-soluble pesticides in the same formulation. An ME is suitable for dilution into water, either remaining as a microemulsion or forming a conventional oil-in-water emulsion.

Suspension concentrates (SC) may comprise aqueous or non-aqueous suspensions of finely divided insoluble solid particles of a compound of the present invention. SCs may be prepared by ball or bead milling the solid compound of the present invention in a suitable medium, optionally with one or more dispersing agents, to produce a fine particle suspension of the compound. One or more wetting agents may be included in the composition and a suspending agent may be included to reduce the rate at which the particles settle. Alternatively, a compound of the present invention may be dry milled and added to water, containing agents hereinbefore described, to produce the desired end product.

Aerosol formulations comprise a compound of the present invention and a suitable propellant (for example n-butane). A compound of the present invention may also be dissolved or dispersed in a suitable medium (for example water or a water miscible liquid, such as n-propanol) to provide compositions for use in non-pressurised, hand-actuated spray pumps.

Capsule suspensions (CS) may be prepared in a manner similar to the preparation of EW formulations but with an additional polymerisation stage such that an aqueous dispersion of oil droplets is obtained, in which each oil droplet is encapsulated by a polymeric shell and contains a compound of the present invention and, optionally, a carrier or diluent therefor. The polymeric shell may be produced by either an interfacial polycondensation reaction or by a coacervation procedure. The compositions may provide for controlled release of the compound of the present invention and they may be used for seed treatment. A compound of the present invention may also be formulated in a biodegradable polymeric matrix to provide a slow, controlled release of the compound.

The composition may include one or more additives to improve the biological performance of the composition, for example by improving wetting, retention or distribution on surfaces; resistance to rain on treated surfaces; or uptake or mobility of a compound of the present invention. Such additives include surface active agents (SFAs), spray additives based on oils, for example certain mineral oils or natural plant oils (such as soy bean and rape seed oil), and blends of these with other bio-enhancing adjuvants (ingredients which may aid or modify the action of a compound of the present invention).

Wetting agents, dispersing agents and emulsifying agents may be SFAs of the cationic, anionic, amphoteric or non-ionic type.

Suitable SFAs of the cationic type include quaternary ammonium compounds (for example cetyltrimethyl ammonium bromide), imidazolines and amine salts.

Suitable anionic SFAs include alkali metals salts of fatty acids, salts of aliphatic monoesters of sulphuric acid (for example sodium lauryl sulphate), salts of sulphonated aromatic compounds (for example sodium dodecylbenzenesulphonate, calcium dodecylbenzenesulphonate, butylnaphthalene sulphonate and mixtures of sodium di-isopropyl- and tri-isopropyl-naphthalene sulphonates), ether sulphates, alcohol ether sulphates (for example sodium laureth-3-sulphate), ether carboxylates (for example sodium laureth-3-carboxylate), phosphate esters (products from the reaction between one or more fatty alcohols and phosphoric acid (predominately mono-esters) or phosphorus pentoxide (predominately di-esters), for example the reaction between lauryl alcohol and tetraphosphoric acid; additionally these products may be ethoxylated), sulphosuccinamates, paraffin or olefine sulphonates, taurates and lignosulphonates.

Suitable SFAs of the amphoteric type include betaines, propionates and glycinates.

Suitable SFAs of the non-ionic type include condensation products of alkylene oxides, such as ethylene oxide, propylene oxide, butylene oxide or mixtures thereof, with fatty alcohols (such as oleyl alcohol or cetyl alcohol) or with alkylphenols (such as octylphenol, nonylphenol or octylcresol); partial esters derived from long chain fatty acids or hexitol anhydrides; condensation products of said partial esters with ethylene oxide; block polymers (comprising ethylene oxide and propylene oxide); alkanolamides; simple esters (for example fatty acid polyethylene glycol esters); amine oxides (for example lauryl dimethyl amine oxide); and lecithins.

Suitable suspending agents include hydrophilic colloids (such as polysaccharides, polyvinylpyrrolidone or sodium carboxymethylcellulose) and swelling clays (such as bentonite or attapulgite).

The compound or composition of the present invention may be applied to a plant, part of the plant, plant organ, plant propagation material or a plant growing locus.

The term “plants” refers to all physical parts of a plant, including seeds, seedlings, saplings, roots, tubers, stems, stalks, foliage, and fruits.

The term “locus” as used herein means fields in or on which plants are growing, or where seeds of cultivated plants are sown, or where seed will be placed into the soil. It includes soil, seeds, and seedlings, as well as established vegetation.

The term “plant propagation material” denotes all generative parts of a plant, for example seeds or vegetative parts of plants such as cuttings and tubers. It includes seeds in the strict sense, as well as roots, fruits, tubers, bulbs, rhizomes, and parts of plants.

The application is generally made by spraying the composition, typically by tractor mounted sprayer for large areas, but other methods such as dusting (for powders), drip or drench can also be used. Alternatively, the composition may be applied in furrow or directly to a seed before or at the time of planting.

The compound or composition of the present invention may be applied pre-emergence or post-emergence. Suitably, where the composition is used to regulate the growth of crop plants or enhance the tolerance to abiotic stress, it may be applied post-emergence of the crop. Where the composition is used to promote the germination of seeds, it may be applied pre-emergence.

The present invention envisages application of the compounds or compositions of the invention to plant propagation material prior to, during, or after planting, or any combination of these.

Although active ingredients can be applied to plant propagation material in any physiological state, a common approach is to use seeds in a sufficiently durable state to incur no damage during the treatment process. Typically, seed would have been harvested from the field; removed from the plant; and separated from any cob, stalk, outer husk, and surrounding pulp or other non-seed plant material. Seed would preferably also be biologically stable to the extent that treatment would not cause biological damage to the seed. It is believed that treatment can be applied to seed at any time between seed harvest and sowing of seed including during the sowing process.

Methods for applying or treating active ingredients on to plant propagation material or to the locus of planting are known in the art and include dressing, coating, pelleting and soaking as well as nursery tray application, in furrow application, soil drenching, soil injection, drip irrigation, application through sprinklers or central pivot, or incorporation into soil (broad cast or in band). Alternatively, or in addition active ingredients may be applied on a suitable substrate sown together with the plant propagation material.

The rates of application of compounds of the present invention may vary within wide limits and depend on the nature of the soil, the method of application (pre- or post-emergence; seed dressing; application to the seed furrow; no tillage application etc.), the crop plant, the prevailing climatic conditions, and other factors governed by the method of application, the time of application and the target crop. For foliar or drench application, the compounds of the present invention according to the invention are generally applied at a rate of from 1 to 2000 g/ha, especially from 5 to 1000 g/ha. For seed treatment the rate of application is generally between 0.0005 and 150 g per 100 kg of seed.

The compounds and compositions of the present invention may be applied to dicotyledonous or monocotyledonous crops. Crops of useful plants in which the composition according to the invention can be used include perennial and annual crops, such as berry plants for examples blackberries, blueberries, cranberries, raspberries and strawberries; cereals for example barley, maize (corn), millet, oats, rice, rye, sorghum triticale and wheat; fibre plants for example cotton, hemp, jute and sisal; field crops for example sugar and fodder beet, coffee, hops, mustard, oilseed rape (canola), poppy, sugar cane, sunflower, tea and tobacco; fruit trees for example apple, apricot, avocado, banana, cherry, citrus, nectarine, peach, pear and plum; grasses for example Bermuda grass, bluegrass, bentgrass, centipede grass, fescue, ryegrass, St. Augustine grass and Zoysia grass; herbs such as basil, borage, chives, coriander, lavender, lovage, mint, oregano, parsley, rosemary, sage and thyme; legumes for example beans, lentils, peas and soya beans; nuts for example almond, cashew, ground nut, hazelnut, peanut, pecan, pistachio and walnut; palms for example oil palm; ornamentals for example flowers, shrubs and trees; other trees, for example cacao, coconut, olive and rubber; vegetables for example asparagus, aubergine, broccoli, cabbage, carrot, cucumber, garlic, lettuce, marrow, melon, okra, onion, pepper, potato, pumpkin, rhubarb, spinach and tomato; and vines for example grapes.

Crops are to be understood as being those which are naturally occurring, obtained by conventional methods of breeding, or obtained by genetic engineering. They include crops which contain so-called output traits (e.g. improved storage stability, higher nutritional value and improved flavour).

Crops are to be understood as also including those crops which have been rendered tolerant to herbicides like bromoxynil or classes of herbicides such as ALS-, EPSPS-, GS-, HPPD- and PPO-inhibitors. An example of a crop that has been rendered tolerant to imidazolinones, e.g. imazamox, by conventional methods of breeding is Clearfield® summer canola. Examples of crops that have been rendered tolerant to herbicides by genetic engineering methods include e.g. glyphosate- and glufosinate-resistant maize varieties commercially available under the trade names RoundupReady®, Herculex I® and LibertyLink®.

Crops are also to be understood as being those which naturally are or have been rendered resistant to harmful insects. This includes plants transformed by the use of recombinant DNA techniques, for example, to be capable of synthesising one or more selectively acting toxins, such as are known, for example, from toxin-producing bacteria. Examples of toxins which can be expressed include δ-endotoxins, vegetative insecticidal proteins (Vip), insecticidal proteins of bacteria colonising nematodes, and toxins produced by scorpions, arachnids, wasps and fungi.

An example of a crop that has been modified to express the Bacillus thuringiensis toxin is the Bt maize KnockOut® (Syngenta Seeds). An example of a crop comprising more than one gene that codes for insecticidal resistance and thus expresses more than one toxin is VipCot® (Syngenta Seeds). Crops or seed material thereof can also be resistant to multiple types of pests (so-called stacked transgenic events when created by genetic modification). For example, a plant can have the ability to express an insecticidal protein while at the same time being herbicide tolerant, for example Herculex I® (Dow AgroSciences, Pioneer Hi-Bred International).

Compounds of the present invention may also be used to promote the germination of seeds of non-crop plants, for example as part of an integrated weed control program. A delay in germination of weed seeds may provide a crop seedling with a stronger start by reducing competition with weeds. Alternatively compounds of the present invention may be used to delay the germination of seeds of crop plants, for example to increase the flexibility of timing of planting for the grower.

Normally, in the management of a crop a grower would use one or more other agronomic chemicals or biologicals in addition to the compound or composition of the present invention. There is also provided a mixture comprising a compound or composition of the present invention, and a further active ingredient.

Examples of agronomic chemicals or biologicals include pesticides, such as acaricides, bactericides, fungicides, herbicides, insecticides, nematicides, plant growth regulators, crop enhancing agents, safeners as well as plant nutrients and plant fertilizers. Examples of suitable mixing partners may be found in the Pesticide Manual, 15th edition (published by the British Crop Protection Council). Such mixtures may be applied to a plant, plant propagation material or plant growing locus either simultaneously (for example as a pre-formulated mixture or a tank mix), or sequentially in a suitable timescale. Co-application of pesticides with the present invention has the added benefit of minimising farmer time spent applying products to crops. The combination may also encompass specific plant traits incorporated into the plant using any means, for example conventional breeding or genetic modification.

The present invention provides the use of a compound of Formula (I), or a composition comprising a compound according to Formula (I) and an agriculturally acceptable formulation adjuvant, for improving the tolerance of a plant to abiotic stress, regulating or improving the growth of a plant, promoting seed germination and/or safening a plant against phytotoxic effects of chemicals.

The present invention also provides the use of a compound, composition or mixture of the present invention, for improving the tolerance of a plant to abiotic stress, regulating or improving the growth of a plant, promoting seed germination and/or safening a plant against phytotoxic effects of chemicals.

Formulation Examples

| Wettable powders | a) | b) | c) |
|---|---|---|---|
| active ingredient [compound of formula (I)] | 25% | 50% | 75% |
| sodium lignosulfonate | 5% | 5% | — |
| sodium lauryl sulfate | 3% | — | 5% |
| sodium diisobutylnaphthalenesulfonate | — | 6% | 10% |
| phenol polyethylene glycol ether | — | 2% | — |
| (7-8 mol of ethylene oxide) | | | |
| highly dispersed silicic acid | 5% | 10% | 10% |
| Kaolin | 62% | 27% | — |

The active ingredient is thoroughly mixed with the adjuvants and the mixture is thoroughly ground in a suitable mill, affording wettable powders that can be diluted with water to give suspensions of the desired concentration.

| Powders for dry seed treatment | a) | b) | c) |
|---|---|---|---|
| active ingredient [compound of formula (I)] | 25% | 50% | 75% |
| light mineral oil | 5% | 5% | 5% |
| highly dispersed silicic acid | 5% | 5% | — |
| Kaolin | 65% | 40% | — |
| Talcum | — | | 20% |

The active ingredient is thoroughly mixed with the adjuvants and the mixture is thoroughly ground in a suitable mill, affording powders that can be used directly for seed treatment.

Emulsifiable concentrate

| | |
|---|---|
| active ingredient [compound of formula (I)] | 10% |
| octylphenol polyethylene glycol ether | 3% |
| (4-5 mol of ethylene oxide) | |
| calcium dodecylbenzenesulfonate | 3% |
| castor oil polyglycol ether (35 mol of ethylene oxide) | 4% |
| Cyclohexanone | 30% |
| xylene mixture | 50% |

Emulsions of any required dilution, which can be used in plant protection, can be obtained from this concentrate by dilution with water.

| Dusts | a) | b) | c) |
|---|---|---|---|
| Active ingredient [compound of formula (I)] | 5% | 6% | 4% |
| talcum | 95% | — | — |
| Kaolin | — | 94% | — |
| mineral filler | — | — | 96% |

Ready-for-use dusts are obtained by mixing the active ingredient with the carrier and grinding the mixture in a suitable mill. Such powders can also be used for dry dressings for seed.

Extruder granules

| | |
|---|---|
| Active ingredient [compound of formula (I)] | 15% |
| sodium lignosulfonate | 2% |
| carboxymethylcellulose | 1% |
| Kaolin | 82% |

The active ingredient is mixed and ground with the adjuvants, and the mixture is moistened with water. The mixture is extruded and then dried in a stream of air.

Coated granules

| | |
|---|---|
| Active ingredient [compound of formula (I)] | 8% |
| polyethylene glycol (mol. wt. 200) | 3% |
| Kaolin | 89% |

The finely ground active ingredient is uniformly applied, in a mixer, to the kaolin moistened with polyethylene glycol. Non-dusty coated granules are obtained in this manner.

Suspension concentrate

| | |
|---|---|
| active ingredient [compound of formula (I)] | 40% |
| propylene glycol | 10% |
| nonylphenol polyethylene glycol ether (15 mol of ethylene oxide) | 6% |
| Sodium lignosulfonate | 10% |
| carboxymethylcellulose | 1% |
| silicone oil (in the form of a 75% emulsion in water) | 1% |
| Water | 32% |

The finely ground active ingredient is intimately mixed with the adjuvants, giving a suspension concentrate from which suspensions of any desired dilution can be obtained by dilution with water. Using such dilutions, living plants as well as plant propagation material can be treated and protected against infestation by microorganisms, by spraying, pouring or immersion.

Flowable concentrate for seed treatment

| | |
|---|---|
| active ingredient [compound of formula (I)] | 40% |
| propylene glycol | 5% |
| copolymer butanol PO/EO | 2% |
| tristyrenephenole with 10-20 moles EO | 2% |
| 1,2-benzisothiazolin-3-one (in the form of a 20% | 0.5% |
| solution in water) | |
| monoazo-pigment calcium salt | 5% |
| Silicone oil (in the form of a 75% emulsion in water) | 0.2% |
| Water | 45.3% |

The finely ground active ingredient is intimately mixed with the adjuvants, giving a suspension concentrate from which suspensions of any desired dilution can be obtained by dilution with water. Using such dilutions, living plants as well as plant propagation material can be treated and protected against infestation by microorganisms, by spraying, pouring or immersion.

### Slow Release Capsule Suspension

28 parts of a combination of the compound of formula (I) are mixed with 2 parts of an aromatic solvent and 7 parts of toluene diisocyanate/polymethylene-polyphenylisocyanate-mixture (8:1). This mixture is emulsified in a mixture of 1.2 parts of polyvinyl alcohol, 0.05 parts of a defoamer and 51.6 parts of water until the desired particle size is achieved. To this emulsion a mixture of 2.8 parts 1,6-diaminohexane in 5.3 parts of water is added. The mixture is agitated until the polymerization reaction is completed.

The obtained capsule suspension is stabilized by adding 0.25 parts of a thickener and 3 parts of a dispersing agent. The capsule suspension formulation contains 28% of the active ingredients. The medium capsule diameter is 8-15 microns.

The resulting formulation is applied to seeds as an aqueous suspension in an apparatus suitable for that purpose.

### EXAMPLES

The Examples which follow serve to illustrate the invention.

### Compound Synthesis and Characterisation

The following abbreviations are used throughout this section: br s=broad singlet, ° C.=degrees Celsius, CDCl₃=chloroform-d, d=doublet, dd=doublet of doublets, m=multiplet, M=molar, MHz=megahertz, s=singlet, sext=sextuplet, THF=tetrahydrofuran
LC-MS were recorded on a Mass Spectrometer from Waters (SQD, SQDII Single quadrupole mass spectrometer) equipped with an electrospray source (Polarity: positive and negative ions, Capillary: 3.00 kV, Cone range: 30 V, Extractor: 2.00 V, Source Temperature: 150° C., Desolvation Temperature: 350° C., Cone Gas Flow: 50 I/h, Desolvation Gas Flow: 650 I/h, Mass range: 100 to 900 Da) and an Acquity UPLC from Waters: Binary pump, heated column compartment, diode-array detector and ELSD detector. Column: Waters UPLC HSS T3, 1.8 μm, 30×2.1 mm, Temp: 60° C., DAD Wavelength range (nm): 210 to 500.
Protocol A: Runtime: 1.5 min; Solvents: A=water+5% MeOH+0.05% HCOOH, B=Acetonitrile+0.05% HCOOH; Flow (ml/min) 0.85, Gradient: 10% B isocratic for 0.2 min, then 10-100% B in 1.0 min, 100% B isocratic for 0.2 min, 100-10% B in 0.05 min, 10% B isocratic for 0.05 min.
Protocol B: Runtime: 3.0 min; Solvents: A=water+5% MeOH+0.05% HCOOH, B=Acetonitrile+0.05% HCOOH; Flow (ml/min) 0.85, Gradient: 10% B isocratic for 0.2 min, then 10-100% B in 2.5 min, 100% B isocratic for 0.3 min. Rₜ stands for retention time.
General Procedure A: This example illustrates the general synthetic preparation of stannane compounds of formula (VI-a)

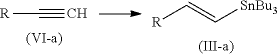

To a solution of terminal alkyne substrate of formula (VI-a) (1.0 eq.) and Pd(PPh₃)₄ (0.05 eq.) in THf (0.2 M), tributyltin hydride (1.2 eq.) was added at room temperature under argon. After the mixture was stirred for 90 min, the solution was filtered over Celite. The resulting solution was concentrated in vacuo. The crude material was purified by flash column chromatography (SiO₂, cyclohexane) to give the corresponding stannane compound of formula (III-a) (80-90% purity) as a colourless oil. The crude stannane material of formula (III-a) was used for the next reaction without further purification.

General Procedure B: This example illustrates the general synthetic preparation of compounds of formula (I-a) via a Stille coupling

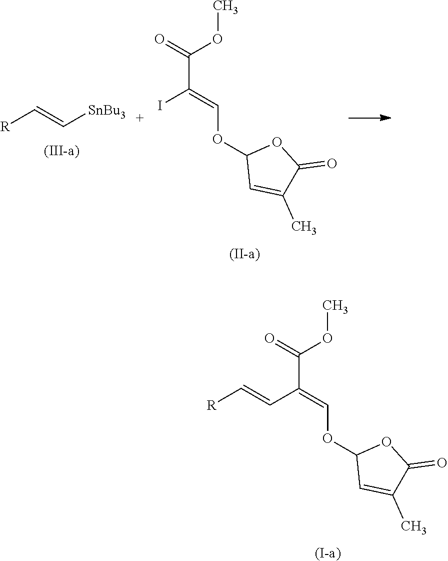

To a solution of vinyl iodide (II-a) (1.0 equiv.), trifurylphosphine (0.40 equiv.) in 1,4-dioxane (0.1 M) was added, followed by the addition of stannane compound of formula (III-a) (3.0 equiv.) under an argon atmosphere. The solution was degassed by bubbling argon through the solution for 10 min. Pd₂(dba)₃ (0.10 equiv.) was added, and then the reaction vial was sealed and heated to 120° C. for 30 min. The mixture solution was allowed to cool to room temperature and filtered over Celite. The solvent was removed in vacuo and the crude material was purified by flash column chromatography (SiO₂, cyclohexane/ethyl acetate), to afford the corresponding coupling product (I-a) as an oil material.

All reactions were performed according to General Procedures A and B unless otherwise stated below.

### Example 1: Methyl (E,2E)-4-(1-methylcyclohexyl)-2-[(4-methyl-5-oxo-2H-furan-2-yl)oxymethylene]but-3-enoate (Compound P-1)

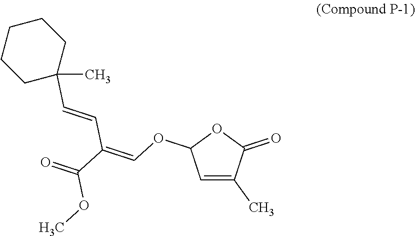 [SMILES: COC(=O)C(/C=C/C1(C)CCCCC1)=C/OC1C=C(C)C(=O)O1]

¹H NMR (400 MHz, CDCl₃) δ ppm: 7.46 (s, 1H), 6.98 (quin, J=1.6 Hz, 1H), 6.42 (d, J=16.9 Hz, 1H), 6.13-6.16 (m, 1H), 6.07 (d, J=16.5 Hz, 1H), 3.76 (s, 3H), 2.04 (t, J=1.5 Hz, 3H), 1.45-1.58 (m, 6H), 1.37-1.43 (m, 2H), 1.33 (m, 2H), 1.02 (s, 3H);

¹³C NMR (101 MHz, CDCl₃) δ ppm: 170.5, 167.1, 150.8, 145.8, 141.3, 135.5, 114.8, 113.0, 100.3, 51.6, 37.8 (2C), 36.6, 27.2, 26.3, 22.4 (2C), 10.7.

### Example 2: Methyl (E,2E)-4-(4,4-difluorocyclohexyl)-2-[(4-methyl-5-oxo-2H-furan-2-yl)oxymethylene]but-3-enoate (Compound P-2)

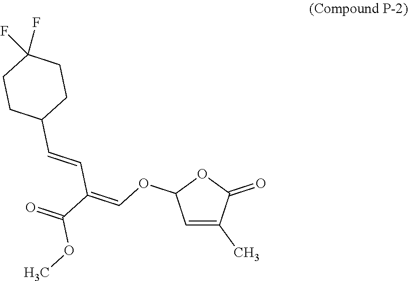 [SMILES: COC(=O)C(/C=C/C1CCC(F)(F)CC1)=C/OC1C=C(C)C(=O)O1]

Purification by reverse phase column chromatography is required;

¹H NMR (400 MHz, CDCl₃) δ ppm: 7.50 (s, 1H), 6.98 (quin, J=1.6 Hz, 1H), 6.36 (dd, J=16.1, 7.3 Hz, 1H), 6.18 (dd, J=16.1, 1.1 Hz, 1H), 6.15 (quin, J=1.4 Hz, 1H), 3.76 (s, 3H), 2.07-2.18 (m, 3H), 2.05 (t, J=1.5 Hz, 3H), 1.67-1.86 (m, 4H), 1.49-1.57 (m, 2H);

¹³C NMR (101 MHz, CDCl₃) δ ppm: 170.4, 166.8, 151.7, 141.2, 138.4, 135.7, 123.3 (t, J=240 Hz), 117.8, 112.1, 100.4, 51.6, 40.0, 33.2 (t, J=24 Hz), 28.7 (d, J=9, 3 Hz), 10.7;

¹⁹F NMR (377 MHz, CDCl₃) δ ppm: −92.05 (d, J=235.7 Hz, 1F), −101.42 (d, J=236.0 Hz, 1F).

### Example 3: Methyl (E,2E)-4-(1-methylcyclopentyl)-2-[(4-methyl-5-oxo-2H-furan-2-yl)oxymethylene]but-3-enoate (Compound P-6)

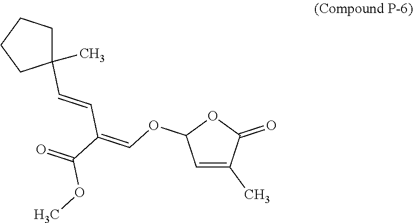 [SMILES: COC(=O)C(/C=C/C1(C)CCCC1)=C/OC1C=C(C)C(=O)O1]

Conditions: Triphenylarsane was used instead of trifurylphosphine. The reaction was stirred at 100° C. for 60 min.

¹H NMR (400 MHz, CD₂Cₗ₂) δ ppm: 7.46 (s, 1H), 7.06-6.93 (m, 1H), 6.50 (d, J=16.5 Hz, 1H), 6.18-6.14 (m, 1H), 6.09 (d, J=16.5 Hz, 1H), 3.73 (s, 3H), 2.00 (s, 2H), 2.04-1.96 (m, 1H), 1.77-1.34 (m, 8H), 1.09 (s, 3H);

¹³C NMR (101 MHz, CD₂Cl₂) δ ppm: 170.5, 166.9, 151.0, 145.2, 141.4, 135.4, 114.1, 112.6, 100.6, 51.4, 45.3, 39.5, 25.2, 24.1, 10.4.

### Example 4: Methyl (E,2E)-4-(1-methylcyclopropyl)-2-[(4-methyl-5-oxo-2H-furan-2-yl)oxymethylene]but-3-enoate (Compound P-7)

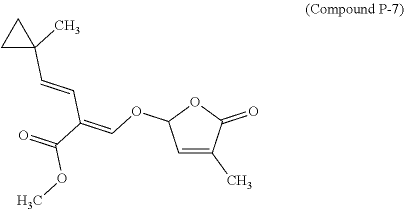 [SMILES: COC(=O)C(/C=C/C1(C)CC1)=C/OC1C=C(C)C(=O)O1]

The reaction was stirred at 100° C. for 30 min; Purification by reverse phase column chromatography is required;

¹H NMR (400 MHz, CDCl₃) δ ppm: 7.43 (s, 1H), 6.97 (quin, J=1.6 Hz, 1H), 6.13-6.16 (m, 1H), 6.09 (d, J=16.2 Hz, 1H), 6.04 (d, J=16.2 Hz, 1H), 3.75 (s, 3H), 2.04 (t, J=1.5 Hz, 3H), 1.22 (s, 3H), 0.64-0.68 (m, 4H);

¹³C NMR (101 MHz, CDCl₃) δ ppm: 170.5, 167.1, 150.3, 143.5, 141.3, 135.6, 114.1, 112.6, 100.4, 51.6, 21.0, 18.3, 15.9 (2C), 10.7.

### Example 5: Methyl (E,2E)-2-[(3,4-dimethyl-5-oxo-2H-furan-2-yl)oxymethylene]-5,5-dimethyl-hex-3-enoate (Compound P-10)

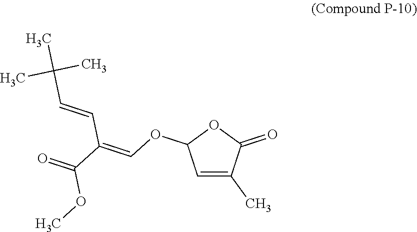 [SMILES: COC(=O)C(/C=C/C(C)(C)C)=C/OC1C=C(C)C(=O)O1]

¹H NMR (400 MHz, CD₂Cl₂)) δ ppm: 7.50 (s, 1H), 6.47 (d, J=16.5 Hz, 1H), 6.10 (d, J=16.5 Hz, 1H), 6.02-5.95 (m, 1H), 3.75 (s, 3H), 2.07-2.03 (m, 3H), 1.91 (t, J=1.1 Hz, 3H), 1.08 (s, 9H);

¹³C NMR (101 MHz, CD₂Cl₂) δ ppm: 170.9, 166.9, 152.9, 151.1, 146.2, 127.8, 113.8, 112.6, 102.1, 51.4, 33.6, 29.1, 11.1, 8.30.

### Example 6: Methyl (E,2E)-4-cyclopentyl-2-[(4-methyl-5-oxo-2H-furan-2-yl)oxymethylene]but-3-enoate (Compound P-11)

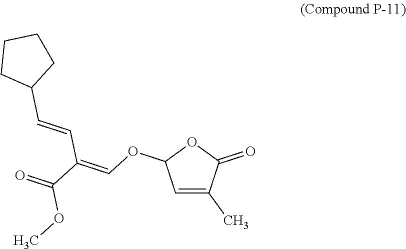 [SMILES: COC(=O)C(/C=C/C1CCCC1)=C/OC1C=C(C)C(=O)O1]

Conditions: Purification by reverse phase column chromatography required.

¹H NMR (400 MHz, CDCl₃) δ ppm: 7.46 (s, 1H), 7.00-6.96 (m, 1H), 6.39 (dd, J=8.1, 16.1 Hz, 1H), 6.18-6.11 (m, 2H), 3.76 (s, 3H), 2.49 (sext, J=8.1 Hz, 1H), 2.06-2.02 (m, 3H), 1.86-1.77 (m, 2H), 1.71-1.63 (m, 2H), 1.62-1.55 (m, 2H), 1.40-1.31 (m, 2H);

¹³C NMR (101 MHz, CDCl₃) δ ppm: 170.5, 167.0, 151.0, 141.3, 140.6, 135.6, 116.7, 112.6, 100.4, 51.6, 44.9, 33.2, 25.3, 10.7.

### Example 7: Methyl (E,2E)-5-methoxy-5-methyl-2-[(4-methyl-5-oxo-2H-furan-2-yl)oxymethylene]hex-3-enoate (Compound P-12)

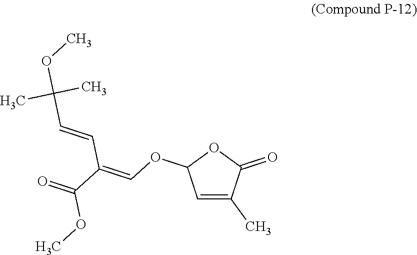 [SMILES: COC(=O)C(/C=C/C(C)(C)OC)=C/OC1C=C(C)C(=O)O1]

¹H NMR (400 MHz, CDCl₃) δ ppm: 7.56 (s, 1H), 7.04-6.93 (m, 1H), 6.41 (d, J=16.9 Hz, 1H), 6.26 (d, J=16.9 Hz, 1H), 6.20-6.09 (m, 1H), 3.78 (s, 3H), 3.19 (s, 3H), 2.05 (s, 3H), 1.32 (s, 6H);

¹³C NMR (101 MHz, CDCl₃) δ ppm: 170.4, 166.9, 152.3, 141.2, 139.9, 135.7, 117.8, 111.8, 100.4, 75.5, 51.7, 50.5, 25.8, 10.7.

### Example 8: Methyl (E,2E)-4-cyclohexyl-2-[(4-methyl-5-oxo-2H-furan-2-yl)oxymethylene]but-3-enoate (Compound P-13)

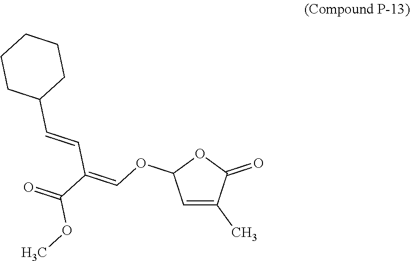 [SMILES: COC(=O)C(/C=C/C1CCCCC1)=C/OC1C=C(C)C(=O)O1]

¹H NMR (400 MHz, CDCl₃) δ ppm: 7.45 (s, 1H), 7.00-6.90 (m, 1H), 6.35 (dd, J=7.2, 16.3 Hz, 1H), 6.14-6.06 (m, 2H), 3.75 (s, 3H), 2.01 (br s, 4H), 1.77-1.62 (m, 5H), 1.31-1.08 (m, 5H);

¹³C NMR (101 MHz, CDCl₃) δ ppm: 170.5, 167.0, 151.1, 141.7, 141.3, 135.6, 116.2, 112.7, 100.4, 51.6, 42.2, 32.8, 26.1, 26.0, 10.7.

### Example 9: Methyl (E,2E)-5,5-dimethyl-2-[(4-methyl-5-oxo-2H-furan-2-yl)oxymethylene]hex-3-enoate (Compound P-14)

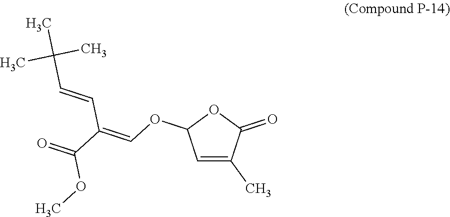 [SMILES: COC(=O)C(/C=C/C(C)(C)C)=C/OC1C=C(C)C(=O)O1]

¹H NMR (400 MHz, CD₂Cl₂) δ ppm: 7.49 (s, 1H), 7.06-6.95 (m, 1H), 6.48 (d, J=16.5 Hz, 1H), 6.19 (s, 1H), 6.09 (d, J=16.5 Hz, 1H), 3.75 (s, 3H), 2.05-2.00 (m, 3H), 1.08 (s, 9H);

¹³C NMR (101 MHz, CD₂Cl₂) δ ppm: 170.5, 166.9, 151.2, 146.2, 141.4, 135.4, 113.6, 112.5, 100.6, 51.4, 33.7, 29.1, 10.4.

TABLE 2
LC/MS data (Rₜ = Retention time) for selected compounds of Table 1.

| | Compound | | |
|---|---|---|---|
| No. | Name | Structure | LC/MS |
| P-1 | methyl (E,2E)-4-(1- methylcyclohexyl)-2-[(4- methyl-5-oxo-2H-furan-2- yl)oxymethylene]but-3- enoate | 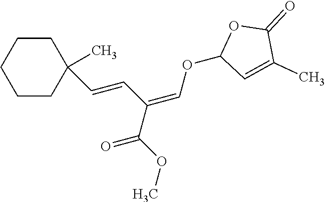 [SMILES: COC(=O)C(/C=C/C1(C)CCCCC1)=C/OC1C=C(C)C(=O)O1] | Rₜ = 1.05 min (A); MS: m/z = 321 (M + H) |
| P-2 | methyl (E,2E)-4-(4,4- difluorocyclohexyl)-2-[(4- methyl-5-oxo-2H-furan-2- yl)oxymethylene]but-3- enoate | 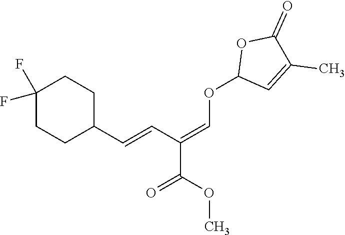 [SMILES: COC(=O)C(/C=C/C1CCC(F)(F)CC1)=C/OC1C=C(C)C(=O)O1] | Rₜ = 1.08 min (A); MS: m/z = 343 (M + H) |
| P-3 | methyl (E,2E)-4- cyclohexyl-2-[(3,4- dimethyl-5-oxo-2H-furan- 2-yl)oxymethylene]but-3- enoate | 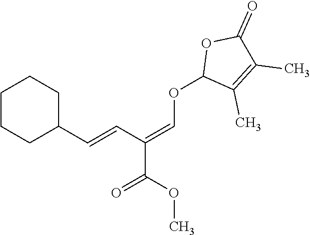 [SMILES: COC(=O)C(/C=C/C1CCCCC1)=C/OC1OC(=O)C(C)=C1C] | Rₜ = 1.98 min (B); MS: m/z = 321 (M + H) |
| P-4 | methyl (E,2E)-4-(1- adamantyl)-2-[(4-methyl-5- oxo-2H-furan-2- yl)oxymethylene]but-3- enoate | 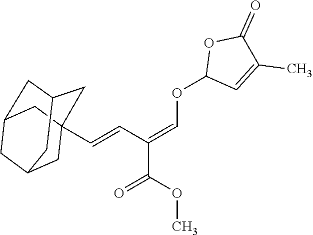 [SMILES: COC(=O)C(/C=C/C12CC3CC(CC(C3)C1)C2)=C/OC1C=C(C)C(=O)O1] | Rₜ = 2.15 min (B); MS: m/z = 359 (M + H) |
| P-5 | methyl (E,2E)-4- cyclobutyl-2-[(4-methyl-5- oxo-2H-furan-2- yl)oxymethylene]but-3- enoate | 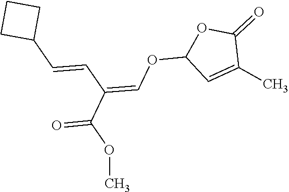 [SMILES: COC(=O)C(/C=C/C1CCC1)=C/OC1C=C(C)C(=O)O1] | Rₜ = 1.69 min (B); MS: m/z = 279 (M + H) |
| P-6 | methyl (E,2E)-4-(1- methylcyclopentyl)-2-[(4- methyl-5-oxo-2H-furan-2- yl)oxymethylene]but-3- enoate | 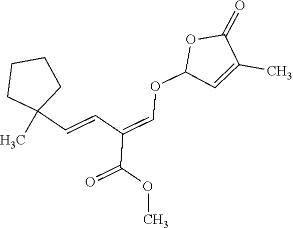 [SMILES: COC(=O)C(/C=C/C1(C)CCCC1)=C/OC1C=C(C)C(=O)O1] | Rₜ = 1.15 min (A); MS: m/z = 307 (M + H) |
| P-7 | methyl (E,2E)-4-(1- methylcyclopropyl)-2-[(4- methyl-5-oxo-2H-furan-2- yl)oxymethylene]but-3- enoate | 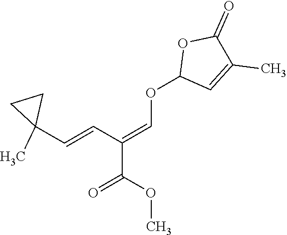 [SMILES: COC(=O)C(/C=C/C1(C)CC1)=C/OC1C=C(C)C(=O)O1] | Rₜ = 1.02 min (A); MS: m/z = 279 (M + H) |
| P-8 | dimethyl (E,4E)-4-[(4- methyl-5-oxo-2H-furan-2- yl)oxymethylene]pent-2- enedioate | 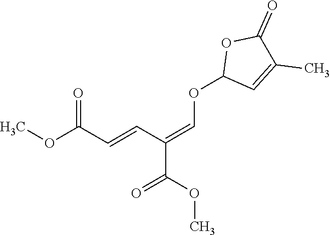 | Rₜ = 0.47 min (B); MS: m/z = not detected |
| P-9 | methyl (2E)-4-methyl-2- [(4-methyl-5-oxo-2H-furan- 2-yl)oxymethylene]pent-3- enoate | 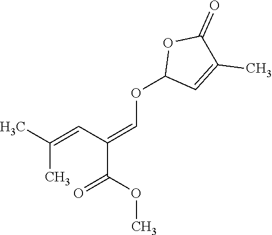 [SMILES: COC(=O)/C(C=C(C)C)=C/OC1C=C(C)C(=O)O1] | Rₜ = 1.33 min (B); MS: m/z = 253 (M + H) |
| P-10 | methyl (E,2E)-2-[(3,4- dimethyl-5-oxo-2H-furan- 2-yl)oxymethylene]-5,5- dimethyl-hex-3-enoate | 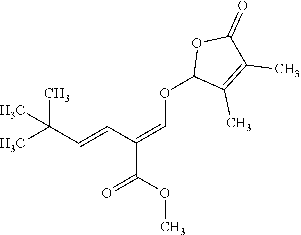 [SMILES: COC(=O)C(/C=C/C(C)(C)C)=C/OC1OC(=O)C(C)=C1C] | Rₜ = 1.10 min (A); MS: m/z = 295 (M + H) |
| P-11 | methyl (2E,3E)-4- cyclopentyl-2-(((4-methyl- 5-oxo-2,5-dihydrofuran-2- yl)oxy)methylene)but-3- enoate | 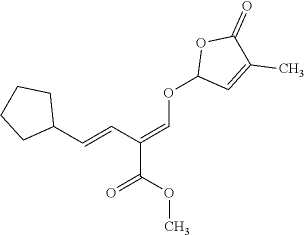 [SMILES: COC(=O)C(/C=C/C1CCCC1)=C/OC1C=C(C)C(=O)O1] | Rₜ = 1.10 min (A); MS: m/z = 293 (M + H) |
| P-12 | methyl (E,2E)-5-methoxy- 5-methyl-2-[(4-methyl-5- oxo-2H-furan-2- yl)oxymethylene]hex-3- enoate | 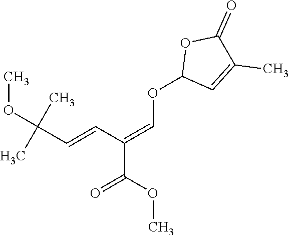 [SMILES: COC(=O)C(/C=C/C(C)(C)OC)=C/OC1C=C(C)C(=O)O1] | Rₜ = 0.90 min (A); MS: m/z = 319 (M + H) |
| P-13 | methyl (E,2E)-4- cyclohexyl-2-[(4-methyl-5- oxo-2H-furan-2- yl)oxymethylene]but-3- enoate | 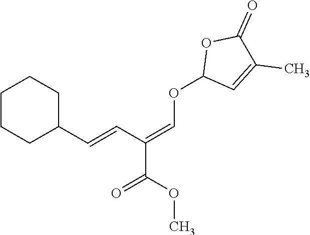 [SMILES: COC(=O)C(/C=C/C1CCCCC1)=C/OC1C=C(C)C(=O)O1] | Rₜ = 1.87 min (B); MS: m/z = 307 (M + H) |
| P-14 | methyl (E,2E)-5,5- dimethyl-2-[(4-methyl-5- oxo-2H-furan-2- yl)oxymethylene]hex-3- enoate | 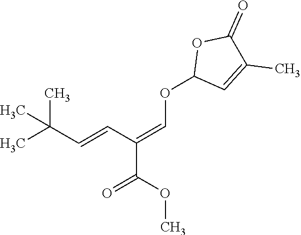 [SMILES: COC(=O)C(/C=C/C(C)(C)C)=C/OC1C=C(C)C(=O)O1] | Rₜ = 1.06 min (A); MS: m/z = 281 (M + H) |
| P-15 | methyl (E,2E)-4-(1- isopropenyl-2,2-dimethyl- 4-oxo-cyclobutyl)-2-[(4- methyl-5-oxo-2H-furan-2- yl)oxymethylene]but-3- enoate | 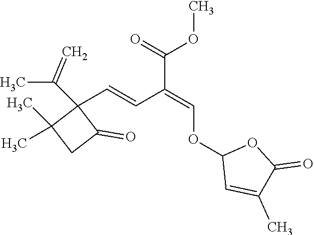 [SMILES: C=C(C)C1(/C=C/C(=C\OC2C=C(C)C(=O)O2)C(=O)OC)C(=O)CC1(C)C] | Rₜ = 1.06 min (A); MS: m/z = 361 (M + H) |
| P-16 | methyl (E,2E)-5-cyano-5- methyl-2-[(4-methyl-5-oxo- 2H-furan-2- yl)oxymethylene]hex-3- enoate | 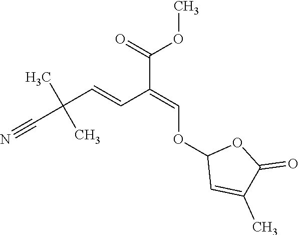 [SMILES: COC(=O)C(/C=C/C(C)(C)C#N)=C/OC1C=C(C)C(=O)O1] | Rₜ = 0.91 min (A); MS: m/z = 292 (M + H) |
| P-17 | methyl (E,2E)-5- [acetyl(methyl)amino]-5- methyl-2-[(4-methyl-5-oxo- 2H-furan-2- yl)oxymethylene]hex-3- enoate | 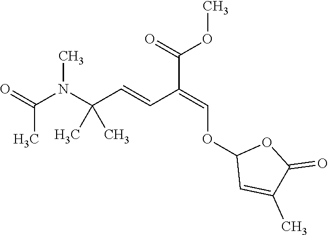 [SMILES: COC(=O)C(/C=C/C(C)(C)N(C)C(C)=O)=C/OC1C=C(C)C(=O)O1] | Rₜ = 0.84 min (A); MS: m/z = 338 (M + H) |
| P-18 | methyl (E,2E)-5-methyl-2- [(4-methyl-5-oxo-2H-furan- 2-yl)oxymethylene]-5- methylsulfonyl-hex-3- enoate | 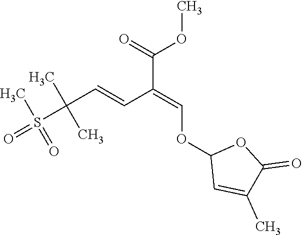 [SMILES: COC(=O)C(/C=C/C(C)(C)S(C)(=O)=O)=C/OC1C=C(C)C(=O)O1] | Rₜ = 0.79 min (A); MS: m/z = not detected |
| P-19 | dimethyl (E,5E)-2,2- dimethyl-5-[(4-methyl-5- oxo-2H-furan-2- yl)oxymethylene]hex-3- enedioate | 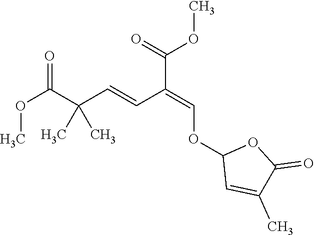 [SMILES: COC(=O)C(/C=C/C(C)(C)C(=O)OC)=C/OC1C=C(C)C(=O)O1] | Rₜ = 0.95 min (A); MS: m/z = 325 (M + H) |
| P-20 | methyl (E,2E)-6- (dimethylamino)-5,5- dimethyl-2-[(4-methyl-5- oxo-2H-furan-2- yl)oxymethylene]-6-oxo- hex-3-enoate | 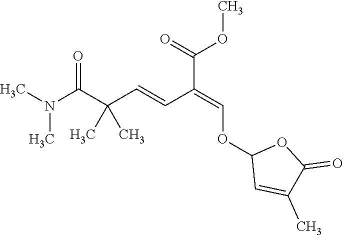 [SMILES: COC(=O)C(/C=C/C(C)(C)C(=O)N(C)C)=C/OC1C=C(C)C(=O)O1] | Rₜ = 0.84 min (A); MS: m/z = 338 (M + H) |

### Biological Examples

### Example B1: Corn Seed Germination Under Cold Stress Conditions

Method: Corn seeds (var. NK Falkone, Syngenta Seeds SAS, St. Sauveur, France) were sorted by size using a sieve to eliminate round seeds. The corn seeds were placed in 24 well plates (1 seed per well, each plate was considered as one experimental unit or replicate). Germination was initiated by the addition of 250 μL of distilled water containing 0.5% DMSO per well (and the tested the substance at the given concentration). 8 replicates (i.e., 8 plates) were used for each treatment characterization. Plates were sealed using seal foil (Polyolefin Art. Nr. 900320) from HJ-BIOANALYTIK. All plates were placed in a climatic chamber at 15° C. with 60% Relative Humidity. The experiment was laid out in a completely randomized design.

Germination was followed over time by taking photographs at different time points. Image analysis was performed automatically with a macro which was developed using the Image J software. A kinetic analysis of germination was carried out by fitting a trend curve. Three parameters were calculated from the trend curve: the Tso (time taken for 50% germination; speed of germination); the slope of the curve (the uniformity of germination) and the plateau (the total percentage of germinated seeds).

The treatments with a given compound were performed at 4 concentrations 2, 10, 50, and 250 μM.

At the highest rate (250 μM), compounds P-2, P-3, P-4, P-6, P-8, P-9, P-10, P-15, and P-19 reduce the Tso value by more than 5% in comparison with untreated control. Moreover, at the same rate (250 μM) compounds P-2, P-3, P-6, P-8, P-9, P-10, P-12, P-15, and P-19 increased the slope of the curve at Tso by more than 50% compared to untreated control.

### Example B2: Dark Induced Senescence of Corn Leaf

It is known that strigolactones regulate (accelerate) leaf senescence, potentially through D14 receptor signaling. It is also known that senescence is an important process allowing reallocation of nutrients. Compounds of formula (I) were tested in a corn leaf dark induced senescence assay and compared to untreated control.

Corn plants of variety Multitop were grown in a greenhouse with relative 75% humidity and at 23-25° C. for 6 weeks. 1.4 cm diameter leaf discs were placed into 24-well plates containing a test compounds in a concentration gradient (100 μM-0.0001 μM) at a final concentration of 0.5% DMSO. Each concentration was tested in 12 replicates. Plates were sealed with seal foil. The foil was pierced to provide gas exchange in each well. The plates were placed into the completely dark climatic chamber. Plates were incubated in the chamber with 75% humidity and at 23° C. for 8 days. On days 0, 5, 6, 7 and 8 photographs were taken of each plate, and image analysis conducted with a macro developed using the ImageJ software. The image analysis was used to determine the concentration at which 50% senescence was achieved (IC50). The lower the value, the higher senescence induction potency. Compounds P-2, P-4, P-6, P-7, P-8, P-12, P-15, P-16, P-17, P-19, and P-20 display an IC₅₀ lower than 3 μM.

1. A compound of formula (I): 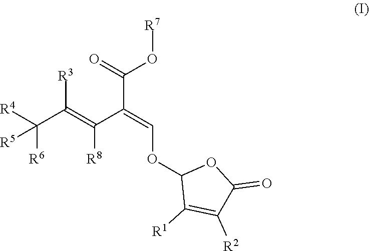
wherein
R¹ is hydrogen, halogen, C₁-C₃alkyl, C₁-C₃alkoxy, or C₃-C₆cycloalkyl;
R² is hydrogen, halogen, C₁-C₃alkyl, C₁-C₃alkoxy, or C₃-C₆cycloalkyl;
R³ is hydrogen or C₁-C₃alkyl;
R⁴ is hydrogen, cyano, C₁-C₆alkyl, C₁-C₆alkoxy, C₁-C₆haloalkyl, C₂-C₆alkenyl, C₂-C₆alkynyl, C₁-C₃alkoxycarbonyl, N,N-di(C₁-C₃alkyl)aminocarbonyl, N,N-di(C₁-C₃alkyl)aminooxycarbonyl, C₁-C₆alkylcarbonyl-N(C₁-C₃alkyl)amino, C₁-C₆alkylsulfinyl, C₁-C₆alkylsulfonyl, or C₁-C₆alkylsulfanyl;
R⁵ is hydrogen, C₁-C₆alkyl, C₁-C₆alkoxy, C₁-C₆haloalkyl, C₂-C₆alkenyl, or C₂-C₆alkynyl;
R⁶ is hydrogen, cyano, C₁-C₆alkyl, C₁-C₆alkoxy, C₁-C₆haloalkyl, C₂-C₆alkenyl, C₂-C₆alkynyl, C₁-C₃alkoxycarbonyl, N,N-di(C₁-C₃alkyl)aminocarbonyl, N,N-di(C₁-C₃alkyl)aminooxycarbonyl, C₁-C₆alkylcarbonyl-N(C₁-C₃alkyl)amino, C₁-C₆alkylsulfinyl, C₁-C₆alkylsulfonyl, or C₁-C₆alkylsulfanyl; or
R⁴ and R⁶ together with the carbon atom to which they are attached form an oxo group or a 3- to 6-membered cycloalkyl ring, optionally substituted with 1, 2, 3, or 4 groups selected from R⁹; or
R⁵ and R⁶ together with the carbon atom to which they are attached form an oxo group or a 3- to 6-membered cycloalkyl ring, optionally substituted with 1, 2, 3, or 4 groups selected from R⁹; or
R⁴, R⁵ and R⁶ together with the carbon atom to which they are attached form an 8- to 12-membered carbotricyclic ring system optionally substituted with 1, 2, 3, or 4 groups selected from R⁹;
R⁷ is hydrogen, C₁-C₆alkyl, C₁-C₆haloalkyl, or C₃-C₆cycloalkyl;
R⁸ is hydrogen or C₁-C₃alkyl;
R⁹ is halogen, C₁-C₃alkyl, C₁-C₃alkoxy, cyano, or oxo (═O);
or a salt thereof.

2. The compound according to claim 1, wherein R¹ is hydrogen, C₁-C₃alkyl, or C₁-C₃alkoxy.

3. The compound according to claim 1, wherein R² is C₁-C₃alkyl, or C₁-C₃alkoxy.

4. The compound according to claim 1, wherein R⁵ is hydrogen, C₁-C₃alkyl, or C₂-C₄alkenyl.

5. The compound according to claim 1, wherein R⁴ is hydrogen, cyano, C₁-C₆alkyl, C₁-C₆alkoxy, C₁-C₆haloalkyl, C₂-C₆alkenyl, C₂-C₆alkynyl, C₁-C₃alkoxycarbonyl, N,N-di(C₁-C₃alkyl)aminocarbonyl, N,N-di(C₁-C₃alkyl)aminooxycarbonyl, C₁-C₃alkylcarbonyl-N(C₁-C₃alkyl)amino, C₁-C₃alkylsulfinyl, C₁-C₃alkylsulfonyl, or C₁-C₃alkylsulfanyl.

6. The compound according to claim 1, wherein R⁶ is hydrogen, cyano, C₁-C₃alkyl, C₁-C₃alkoxy, C₁-C₃alkoxycarbonyl, N,N-di(C₁-C₃alkyl)aminooxycarbonyl, C₁-C₃alkylsulfonyl, or C₁-C₃alkylsulfanyl.

7. The compound according to claim 1, wherein R⁴ and R⁶ together with the carbon atom to which they are attached form a 4- to 6-membered cycloalkyl ring optionally substituted with 1, 2, or 3 groups selected from R⁹.

8. The compound according to claim 1, wherein R⁴, R⁵ and R⁶ together with the carbon atom to which they are attached form an 8- to 10-membered carbotricyclic ring system optionally substituted with 1 or 2 groups selected from R⁹.

9. The compound according to claim 1, wherein R⁷ is C₁-C₃alkyl.

10. A plant growth regulating or seed germination promoting composition, comprising the compound according to claim 1, and an agriculturally acceptable formulation adjuvant.

11. A method for regulating the growth of plants at a locus, said method comprising applying to the locus a compound according to claim 1.

12. A method for promoting the germination of seeds, comprising applying to the seeds, or a locus containing the seeds, the compound according to claim 1.

13. A method for improving the nutrient uptake of a crop, comprising applying to the plant or locus thereof, the compound according to claim 1.

14. Use of a compound of Formula (I) according to claim 1 for promoting the germination of seeds and/or for regulating plant growth.

15. A seed comprising a compound of Formula (I) according to claim 1.
